(19) United States
(12) Patent Application Publication
(10) Pub. No.: US 2021/0183519 A1
(43) Pub. Date: Jun. 17, 2021

(54) METHODS AND SYSTEMS FOR ENHANCING SAFETY OF PSYCHEDELIC DRUG THERAPIES

(71) Applicant: Eleusis Benefit Corporation, PBC, New York, NY (US)

(72) Inventors: Shlomi RAZ, Brooklyn, NY (US); Neiloufar FAMILY, Brooklyn, NY (US)

(21) Appl. No.: 16/757,301

(22) PCT Filed: Oct. 19, 2018
(86) PCT No.: PCT/US18/56746

§ 371 Date: Apr. 17, 2020

Related U.S. Application Data
(60) Provisional application No. 62/574,307, filed on Oct. 19, 2017.

(51) Int. Cl. G16H 50/30; G16H 20/10; G16H 10/20

(52) U.S. Cl. CPC G16H 50/30; G16H 10/20; G16H 20/10

(57) ABSTRACT

The present invention provides methods and systems of enhancing the safety of psychedelic drug therapies (e.g., 5-HT2A agonists (e.g., LSD and psilocybin), dissociatives, and empathogens), for example, as part of a complex therapy. In particular, the invention features methods and systems for reducing the risk of developing psychosis, hypomania, or mania associated with psychedelic therapy.

### BACKGROUND OF THE INVENTION

Psychedelic, dissociative, and empathogenic compounds have demonstrated therapeutic effect in a wide variety of clinical applications, including depression, anxiety, substance abuse, and a variety of other conditions. In addition, psychedelic compounds have also demonstrated therapeutic effect in inflammation related disease, at both perceptual and sub-perceptual dose levels. The therapeutic utility of psychedelic compounds provides the impetus for the development of supportive technologies to enhance the safety and efficacy of the treatment of conditions with these compounds.

A significant clinical risk associated with the therapeutic application of psychedelic, dissociative, and empathogenic compounds is the precipitation of psychosis, hypomania, or mania in patients prone to these conditions. In modern application, this risk is mitigated through patient screening, which utilizes structured interviews administered by clinicians to exclude patients with either a personal history or family history of psychotic-spectrum disorders or associated symptoms. Methods that enhance or replace this approach are required to further enhance patient safety and create more scalable approaches to screening that do not necessitate clinician involvement. In addition, new approaches to the ongoing monitoring of patient safety, and specifically, to the monitoring and mitigation of risk in the days and weeks following treatment with psychedelic therapy are required for broad adoption of psychedelic therapy in the context of public health.

Repeated and chronic administration of psychedelic compounds on an out-patient basis for the treatment of inflammatory or other conditions necessitates a new approach to ongoing monitoring of patient safety. The risks associated with the chronic use of psychedelic compounds, for example, at low-to-sub perceptual doses, are not fully elucidated. However, given concerns regarding the precipitation of psychosis, hypomania, or mania associated with psychedelic compounds, new approaches to enhance patient safety and providing unobtrusive clinical monitoring for psychiatric complications are required.

### SUMMARY OF THE INVENTION

The present invention provides enhanced patient safety through unobtrusive monitoring with the capacity to alert clinicians to the emergence of psychotic-spectrum disorders related to (i) long-term low-to-sub perceptual use of psychedelic compounds and/or (ii) acute medium-to-high dose use of psychedelic compounds. In particular, the invention provides an adjunctive use of a mobile health software application and supportive infrastructure to enhance patient safety in therapeutic regimens involving treatment with psychedelic agents (e.g., 5-HT_{2A} agonists), dissociatives (e.g., esketamine, NMDA receptor antagonists, e.g., ketamine or esketamine), empathogens (e.g., 3,4-methylenedioxymethamphetamine (MDMA)), and other psychoactive compounds that have been associated with the risk of inducing psychosis, hypomania, or mania.

In one aspect, the invention features a method of screening a candidate for treatment with a psychedelic agent. The method includes (i) obtaining a language sample from a treatment candidate; (ii) deriving one or more language characteristics from the language sample; and (iii) based on the one or more language characteristics, determining a measure of risk. The measure of risk correlates with a risk of precipitating or exacerbating psychosis, hypomania, or mania in the candidate. In some embodiments, the method further includes sending a report to a third party. The third party can be, for example, a clinical professional (e.g., a physician, pharmacist, administrative professional, nurse, support professional, or caretaker). In other embodiments, the third party can be a computing platform (e.g., a computer database accessible to one or more clinical professionals, such as pharmacy staff, who may access the computing platform to obtain instructions to fill a psychedelic prescription or not). Thus, in some embodiments, the report informs a decision to prescribe or administer the psychedelic therapy. For example, if the risk of precipitating or exacerbating psychosis, hypomania, or mania is above a predetermined threshold or a reference value, the report instructs a third party that a the psychedelic therapy should not be prescribed or administered. In some embodiments, the report informs a dosing regimen for the psychedelic therapy. For example, if the risk of precipitating or exacerbating psychosis, hypomania, or mania is below a predetermined threshold or a reference value, the report instructs a third party to increase the dose of psychedelic agent. Conversely, if the risk of precipitating or exacerbating psychosis, hypomania, or mania is above a predetermined threshold or a reference value, the report instructs a third party to decrease the dose of psychedelic agent.

In some embodiments of any of the methods described herein, the candidate or patient has been characterized as unlikely to have or develop a paranoid ideation or propensity toward paranoid thinking, paranoid personality disorder, a personality disorder, an intellectual disability (e.g., intellectual developmental disorder), or bipolar disorder. In some embodiments, any of the methods of the invention include screening the candidate for a likelihood of having or developing a paranoid ideation or propensity toward paranoid thinking, paranoid personality disorder, a personality disorder, an intellectual disability (e.g., intellectual developmental disorder), or bipolar disorder. Methods of screening for such disorders and characteristics can be adapted for the present invention from methods known in the art, such as industry-standard questionnaires. In some embodiments, such screening methods can be conducted by a clinician (e.g., in person). Additionally or alternatively, screening methods can be conducted using a mobile device configured to perform any one or more of the methods provided herein.

In another aspect, the invention features a method of reducing a risk of developing psychosis, hypomania, or mania in a patient undergoing treatment with a psychedelic agent, the method including: (i) obtaining a language sample from the patient undergoing treatment with a psychedelic agent; (ii) deriving one or more characteristics of the language sample; (iii) based on the one or more characteristics, determining a measure of risk, wherein the measure of risk correlates with the risk of precipitating or exacerbating psychosis, hypomania, or mania in the patient; and (iv) based on the measure of risk, recommending whether to suspend the treatment with the psychedelic agent (e.g., as part of a report sent to a third party). Thus, the method further includes sending a report to a third party. The third party can be, for example, a clinical professional (e.g., a physician, pharmacist, administrative professional, nurse, support professional, or caretaker). In other embodiments, the third party can be a computing platform (e.g., a computer database accessible to one or more clinical professionals, such as pharmacy staff, who may access the computing platform to obtain instructions to fill a psychedelic prescription or not). Thus, in some embodiments, the report informs a decision to prescribe or administer the psychedelic therapy. For example, if the risk of precipitating or exacerbating psychosis, hypomania, or mania is above a predetermined threshold or a reference value, the report instructs a third party that the psychedelic therapy should not be prescribed or administered.

In another aspect, the invention provides a method of assessing a risk of precipitating or exacerbating psychosis, hypomania, or mania in a patient undergoing treatment with a psychedelic agent, the method including: (i) providing a plurality of measures of risk, each measure of risk derived from one or more characteristics of a language sample obtained from the patient, wherein each measure of risk is associated with a different treatment time point (e.g., daily, every 2 days, every 3 days, every 4 days, every 5 days, weekly, monthly, twice per month, twice per week, or three times per week); and (ii) comparing two or more of the plurality of measure of risk (e.g., consecutive or non-consecutive (e.g., latest-to-earliest time point)) to obtain a differential measure of risk, wherein the patient is identified as at risk of precipitating or exacerbating psychosis, hypomania, or mania if the differential measure of risk exceeds a predetermined threshold. The method further includes sending a report to a third party. The third party can be, for example, a clinical professional (e.g., a physician, pharmacist, administrative professional, nurse, support professional, or caretaker). In other embodiments, the third party can be a computing platform (e.g., a computer database accessible to one or more clinical professionals, such as pharmacy staff, who may access the computing platform to obtain instructions to fill a psychedelic prescription or not). Thus, in some embodiments, the report informs a decision to prescribe or administer the psychedelic therapy. For example, if the differential risk of precipitating or exacerbating psychosis, hypomania, or mania is above a predetermined threshold or a reference value, the report instructs a third party that a the psychedelic therapy should not be prescribed or administered.

In another aspect, the invention features a method of providing a regimen of psychedelic therapy to a patient, the method including: (i) providing a differential measure of risk obtained by comparing two or more measures of risk, each measure of risk derived from one or more language characteristics of a language sample obtained from the patient, wherein the one or more measures of risk correlates with the risk of precipitating or exacerbating psychosis, hypomania, or mania in the patient, and wherein each measure of risk is associated with a different treatment time point; and (ii) suspending the psychedelic therapy if the differential measure of risk exceeds a predetermined threshold.

In some embodiments of any of the methods described herein, the patient has been screened for one or more adverse effects associated with the psychedelic agent (e.g., using screening methods known in the art). Additionally or alternatively, a method of the invention may include screening the patient for one or more adverse effects associated with the psychedelic agent. Adverse effects that can be screened for include, e.g., depersonalization, dissociation, derealization, hallucinogenic or psychoactive abuse, hallucinogen-use disorders, hallucinogen-induced disorders, high-risk behaviors, and mania. Methods of screening for such disorders and characteristics can be adapted for the present invention from methods known in the art, such as industry-standard questionnaires. In some embodiments, such screening methods can be conducted by a clinician (e.g., in person). Additionally or alternatively, screening methods can be conducted using a mobile device configured to perform any one or more of the methods provided herein.

In some embodiments, the method further includes administering the psychedelic agent (or recommending administration of the psychedelic agent) if the screening indicates that the patient is not experiencing the one or more adverse effects (e.g., presently experiencing one or more adverse effects or has experienced one or more adverse effects during the course of psychedelic treatment), or if the screening does not indicate that the patient is experiencing the one or more adverse effects (e.g., presently experiencing one or more adverse effects or has experienced one or more adverse effects during the course of psychedelic treatment).

Methods of the invention additionally provide means for determining whether the patient is complying with the prescribed regimen of psychedelic therapy. In some embodiments, the method further includes assessing a measure of compliance with the psychedelic agent. In some embodiments, the method further includes assessing a measure of abuse of the psychedelic agent. Measures of compliance and/or abuse can be derived from one or more digital readouts using the methods and systems of the invention, e.g., by observing a level of a biomarker, for example, a level of a target molecule present in a body sample obtained from the patient (e.g., a level of the psychedelic agent, a metabolite of the psychedelic agent, or another molecule that correlates positively or negatively with the level of the psychedelic agent in the patient).

In some embodiments, methods of the invention include determining a frequency of retreatment of the patient with the psychedelic agent. The frequency of retreatment can be determined by (i) providing a measure of efficacy correlated with a positive therapeutic response in the patient to the psychedelic agent; (ii) providing a measure of risk correlated with a risk of precipitating or exacerbating a disease state associated with stress or a psychopathology; and (iii) based on steps (i) and (ii) (e.g., weighing the measure of risk against the measure of efficacy), determining a frequency of retreatment with the psychedelic agent. The measure of efficacy, the measure of risk, or both, can be output from (and/or confirmed by) a clinical assessment, e.g., using a software configured to communicate with a mobile device or any of the methods or systems described herein (e.g., wherein one or more factors of the clinical assessment include a language characteristic, a behavioral characteristic, and/or a biomarker), or directly by a clinician using known methods, such as industry-standard questionnaires. In some embodiments, the frequency of retreatment is from bi-weekly to annually (e.g., bi-weekly, monthly, four times per year, twice annually, or annually). In some embodiments, a patient is retreated or redosed (e.g., to adjust the amount per dose or frequency of dosing) upon detecting a deterioration in mental health. For example, a patient that is undergoing treatment or has been treated for any of the diseases or disorders described herein can be retreated or redosed for the disease or disorder upon detection of an increase in one or more symptoms associated with the disease or disorder. The detection can be by any of the methods described herein, for example, by obtaining a language characteristic, a behavioral characteristic, or a digital biomarker indicative of the disease or disorder (e.g., through a digital clinical assessment).

In some embodiments, the method of the invention include adjusting the dose and/or frequency of treatment with the psychedelic agent based on one or more of any of the behavioral characteristics, language characteristics, and/or biomarkers described herein, or any of the measures of risk, compliance, or abuse described herein. In some embodiments, the dose is increased (e.g., to address a low measure of efficacy or a high measure of risk). In other embodiments, the dose is decreased (e.g., to decrease a measure of risk when a measure of efficacy indicates that the treatment is working, or to address a high level of one or more biomarkers).

In another aspect, the invention features a method of administering a psychedelic agent to a patient in need thereof, the method including: (i) obtaining one or more measures of risk derived from one or more language characteristics of a language sample obtained from the patient, wherein the one or more measures of risk correlates with the risk of precipitating or exacerbating psychosis, hypomania, or mania in the patient; and (ii) administering the psychedelic agent if the measure of risk is below a predetermined threshold.

In another aspect, the invention features a method of characterizing the influence of a psychedelic agent on the perception of a patient administered therewith, the method including: (i) obtaining a language sample from the patient; (ii) providing one or more language characteristics of the language sample; and (ii) based on the one or more language characteristics, determining a measure of psychedelic influence, wherein the measure of psychedelic influence correlates with the influence of the psychedelic compound on the perception of the patient.

A method of any of the preceding aspects may further include, in response to determining that a candidate has a high measure of risk, prompting an ecological momentary assessment (EMA) of the candidate, e.g., to confirm or further inform a decision regarding a clinical path forward.

In some embodiments of any of the preceding aspects, the language sample is elicited by a digital prompt, a questionnaire, a clinician administered interview. In some embodiments, the language sample may be, or may be obtained from a dream report, a description of a picture, a thematic apperception test, or a neutral text reading. In some embodiments, the language sample is obtained by passive acquisition (e.g., constant or arbitrary monitoring of outgoing audio data or text data). In some embodiments, the language sample is a text sample and/or an audio sample. In some embodiments, the audio sample is analyzed to derive the one or more language characteristics, wherein the one or more language characteristics comprises one or more acoustic features (e.g., a measure of irregular pitch (e.g., standard variance of pitch), zero-crossing rate, kurtosis energy, harmonics-to-noise ratio (HNR), mel-frequency cepstral coefficients (MFCC), and frame energy). In some embodiments, the audio sample is transcribed into text.

In some embodiments, the language sample is analyzed to derive the one or more language characteristics, wherein the one or more language characteristics comprises a measure of semantic coherence. A low measure of semantic coherence may be positively correlated with the risk of developing psychosis. In some embodiments, the one or more language characters include a measure of syntactic complexity. A low measure of syntactic complexity may be positively correlated with the risk of developing psychosis. In some embodiments, the one or more language characters include a measure of maximum phrase length. A low measure of maximum phrase length may be positively correlated with the risk of developing psychosis. In some embodiments, the one or more language characters include a measure of lexicon breadth and/or lexicon depth. A low measure of lexicon breadth or depth may be positively correlated with the risk of developing psychosis. In some embodiments, the one or more language characters include a measure of logorrhea. A high measure of logorrhea may be positively correlated with the risk of developing hypomania or mania. In some embodiments, the one or more language characters include a measure of psychometrics (e.g., latent inhibition). In some embodiments, the one or more language characters include a measure of flight of thought. A high measure of flight of thought may be positively correlated with the risk of developing hypomania or mania. In some embodiments, the one or more language characters include a measure of recursiveness. A high measure of recursiveness may be positively correlated with the risk of developing hypomania or mania.

In some embodiments, the language sample is analyzed to derive speech graph attributes. The speech graph attributes can be obtained for all or a portion of the words used in the speech sample as an input, for example, to a machine learning algorithm.

In any of the preceding aspects, one or more behavioral characteristics further informs the measure of risk. In some embodiments, the one or more behavioral characteristics are derived from a telephone record. For example, the one or more behavioral characteristics derived from a telephone record may be a number or frequency of outgoing calls or messages, a number or frequency of incoming calls or messages, a ratio between a number or frequency of outgoing calls or messages and a number or frequency of incoming calls or messages, a duration of one or more calls, a length of one or more messages, a number of new phone numbers, a number of changes in cell tower IDs, or a number of unique cell tower IDs.

In some embodiments, a number or frequency of outgoing calls or messages is positively correlated with the risk of developing hypomania or mania. In some embodiments, a ratio between a number or frequency of outgoing calls or messages and a number or frequency of incoming calls or messages is positively correlated with the risk of developing hypomania or mania. In some embodiments, a duration of one or more calls is positively correlated with the risk of developing hypomania or mania. In some embodiments, the length of one or more messages is positively correlated with the risk of developing hypomania or mania. In some embodiments, a number of new phone numbers is positively correlated with the risk of developing hypomania or mania.

In some embodiments, the one or more behavioral characteristics include a number or frequency of instances in which a mobile device screen is turned on.

In some instances, the one or more behavioral characteristics include a measure of activity detected by a sensor (e.g., an antenna on a mobile device, e.g., a smartphone). For example, the sensor may be in communication with a global positioning system (GPS). In some embodiments, the measure of activity is a measure of mobility (i.e., change in geographical location, e.g., as monitored by GPS). In some embodiments, a high measure of mobility is positively correlated with the risk of developing hypomania or mania.

In some embodiments, the sensor is an accelerometer (e.g., as part of the mobile device). In some embodiments, the measure of activity comprises a measure of movement. In some embodiments, the measure of movement is positively correlated with the risk of developing psychosis, hypomania, or mania. In some embodiments, the sensor is or is in communication with a wireless network hub (e.g., Amazon Alexa or Google Home). Any behavioral characteristics detectable by the wireless network hub can be relayed to the systems of the present invention and can thus be incorporated into the methods provided herein.

In some embodiments, the measure of movement is a speed of typing.

In some embodiments, a behavioral characteristic describes a patient's behavior on a computer or mobile device, such as a phone. For example, a behavioral characteristic can be derived from one or more human computer interactions (e.g., swipes, taps, and keystroke events) or combination or pattern thereof, e.g., as described in Dagum, npj Digital Medicine 2018, 1(10): 58-70, which is incorporated herein by reference in its entirety.

In some embodiments, the one or more behavioral characteristics are derived from a frequency, duration, or quality of sleep. For example, the measure of frequency, duration, or quality of sleep can be derived from a frequency and/or duration of light exposure (e.g., by a light sensor on a mobile device or any device in communication with a wireless network hub), frequency or overall quantity of movement detected from movement sensors, or activity levels obtained from any other sensor described here (e.g., mobile device usage, such as on-screen time).

In another aspect, the invention features a method of monitoring a psychedelic agent's effect on a patient's perception, for example, to inform a safe time of release from a supervised facility. Provided herein is a method of characterizing the influence of a psychedelic agent on the perception of a patient administered therewith, the method including: (i) obtaining a language sample from the patient; (ii) providing one or more language characteristics of the language sample; and (ii) based on the one or more language characteristics, determining a measure of psychedelic influence, wherein the measure of psychedelic influence correlates with the influence of the psychedelic therapy on the perception of the patient. In some embodiments, the psychedelic agent is administered on an in-patient basis. In such instances, the psychedelic agent may be administered in a perceptible dose. In other embodiments, the psychedelic agent is administered on an out-patient basis, and the psychedelic agent may be administered in a sub-perceptible dose or a perceptible dose. In some embodiments, the method further comprises providing a notification based on the influence of a psychedelic agent on the perception of the patient. In some embodiments, the notification informs a clinician's decision of when drug-induced alterations in perception and cognition of a patient receiving treatment involving a psychedelic agent have returned to baseline or to a sufficiently low level. In some embodiments, the language sample is analyzed to derive the one or more language characteristics, wherein the one or more language characteristics comprises a measure of semantic proximity to one or more dimensions or facets related to an influence of a psychedelic agent (e.g., as described in the 5D-ASC rating scale). In some embodiments, a measure of semantic proximity to one or more concepts related to an influence of a psychedelic agent is positively correlated with the influence of the psychedelic therapy on the perception of the patient.

In another aspect, the invention provides a method of screening a candidate for treatment with a psychedelic agent, the method including: (i) obtaining a behavioral sample from a candidate (e.g., a candidate who has not begun a regimen involving psychedelic therapy); (ii) deriving one or more behavioral characteristics from the behavioral sample; and (iii) based on the one or more behavioral characteristics, determining a measure of risk, wherein the measure of risk correlates with a risk of precipitating or exacerbating psychosis, hypomania, or mania in the candidate. In some embodiments, the method further includes sending a report to a third party. The third party can be, for example, a clinical professional (e.g., a physician, pharmacist, administrative professional, nurse, support professional, or caretaker). In other embodiments, the third party can be a computing platform (e.g., a computer database accessible to one or more clinical professionals, such as pharmacy staff, who may access the computing platform to obtain instructions to fill a psychedelic prescription or not). Thus, in some embodiments, the report informs a decision to prescribe or administer the psychedelic therapy. For example, if the risk of precipitating or exacerbating psychosis, hypomania, or mania is above a predetermined threshold or a reference value, the report instructs a third party that a the psychedelic therapy should not be prescribed or administered. In some embodiments, the report informs a dosing regimen for the psychedelic therapy. For example, if the risk of precipitating or exacerbating psychosis, hypomania, or mania is below a predetermined threshold or a reference value, the report instructs a third party to increase the dose of psychedelic agent. Conversely, if the risk of precipitating or exacerbating psychosis, hypomania, or mania is above a predetermined threshold or a reference value, the report instructs a third party to decrease the dose of psychedelic agent.

In another aspect, the invention features a method of reducing a risk of developing psychosis, hypomania, or mania in a patient undergoing treatment with a psychedelic agent, the method including: (i) obtaining a behavioral sample from the patient undergoing treatment with a psychedelic agent; (ii) deriving one or more characteristics of the behavioral sample; (iii) based on the one or more characteristics, determining a measure of risk, wherein the measure of risk correlates with the risk of precipitating or exacerbating psychosis, hypomania, or mania in the patient; and (iv) based on the measure of risk, recommending whether to suspend the treatment with a psychedelic agent. In some embodiments, the method further includes sending a report to a third party. The third party can be, for example, a clinical professional (e.g., a physician, pharmacist, administrative professional, nurse, support professional, or caretaker). In other embodiments, the third party can be a computing platform (e.g., a computer database accessible to one or more clinical professionals, such as pharmacy staff, who may access the computing platform to obtain instructions to fill a psychedelic prescription or not). Thus, in some embodiments, the report informs a decision to prescribe or administer the psychedelic therapy. For example, if the risk of precipitating or exacerbating psychosis, hypomania, or mania is above a predetermined threshold or a reference value, the report instructs a third party that a the psychedelic therapy should not be prescribed or administered.

In another aspect, the invention provides a method of assessing a risk of precipitating or exacerbating psychosis, hypomania, or mania in a patient undergoing treatment with a psychedelic agent, the method including: (i) providing a plurality of measures of risk, each measure of risk derived from one or more characteristics of a behavioral sample obtained from the patient, wherein each measure of risk is associated with a different treatment time point (e.g., daily, every 2 days, every 3 days, every 4 days, every 5 days, weekly, monthly, twice per month, twice per week, or three times per week); and (ii) comparing two or more of the plurality of measures of risk (e.g., consecutive or non-consecutive (e.g., latest-to-earliest time point)) to obtain a differential measure of risk, wherein the patient is identified as at risk of precipitating or exacerbating psychosis, hypomania, or mania if the differential measure of risk exceeds a predetermined threshold. The method further includes sending a report to a third party. The third party can be, for example, a clinical professional (e.g., a physician, pharmacist, administrative professional, nurse, support professional, or caretaker). In other embodiments, the third party can be a computing platform (e.g., a computer database accessible to one or more clinical professionals, such as pharmacy staff, who may access the computing platform to obtain instructions to fill a psychedelic prescription or not). Thus, in some embodiments, the report informs a decision to prescribe or administer the psychedelic therapy. For example, if the differential risk of precipitating or exacerbating psychosis, hypomania, or mania is above a predetermined threshold or a reference value, the report instructs a third party that a the psychedelic therapy should not be prescribed or administered.

In another aspect, the invention features a method of providing a regimen of psychedelic therapy to a patient, the method including: (i) providing a differential measure of risk obtained by comparing two or more measures of risk, each measure of risk derived from one or more behavioral characteristics of a behavioral sample obtained from the patient, wherein the one or more measures of risk correlates with the risk of precipitating or exacerbating psychosis, hypomania, or mania in the patient, and wherein each measure of risk is associated with a different treatment time point; and (ii) suspending the psychedelic therapy if the differential measure of risk exceeds a predetermined threshold.

In another aspect, the invention features a method of administering a psychedelic agent to a patient in need thereof, the method including: (i) obtaining one or more measures of risk derived from one or more behavioral characteristics of a behavioral sample obtained from the patient, wherein the one or more measures of risk correlates with the risk of precipitating or exacerbating psychosis, hypomania, or mania in the patient; and (ii) administering the psychedelic agent if the measure of risk is below a predetermined threshold.

A method of any of the preceding aspects may further include, in response to determining that a candidate has a high measure of risk, prompting an ecological momentary assessment (EMA) of the candidate, e.g., to confirm or further inform a decision regarding a clinical path forward.

In some embodiments of any of the preceding aspects, the one or more behavioral characteristics are derived from a telephone record. For example, the one or more behavioral characteristics derived from a telephone record may be a number or frequency of outgoing calls or messages, a number or frequency of incoming calls or messages, a ratio between a number or frequency of outgoing calls or messages and a number or frequency of incoming calls or messages, a duration of one or more calls, a length of one or more messages, a number or frequency of newly added contacts, a number of changes in cell tower IDs, or a number of unique cell tower IDs.

In some embodiments, a number or frequency of outgoing calls or messages is positively correlated with the risk of developing hypomania or mania. In some embodiments, a ratio between a number or frequency of outgoing calls or messages and a number or frequency of incoming calls or messages is positively correlated with the risk of developing hypomania or mania. In some embodiments, a duration of one or more calls is positively correlated with the risk of developing hypomania or mania. In some embodiments, the length of one or more messages is positively correlated with the risk of developing hypomania or mania. In some embodiments, a number of new phone numbers is positively correlated with the risk of developing hypomania or mania.

In some embodiments, the one or more behavioral characteristics include a number or frequency of instances in which a mobile device screen is turned on.

In some instances, the one or more behavioral characteristics include a measure of activity detected by a sensor (e.g., an antenna on a mobile device, e.g., a smartphone). For example, the sensor may be in communication with GPS. In some embodiments, the measure of activity is a measure of mobility (i.e., change in geographical location, e.g., as monitored by GPS). In some embodiments, a high measure of mobility is positively correlated with the risk of developing hypomania or mania.

In some embodiments, the sensor is an accelerometer (e.g., as part of the mobile device). In some embodiments, the measure of activity comprises a measure of movement. In some embodiments, the measure of movement is positively correlated with the risk of developing psychosis, hypomania, or mania. In some embodiments, the sensor is or is in communication with a wireless network hub (e.g., Amazon Alexa or Google Home). Any behavioral characteristics detectable by the wireless network hub can be relayed to the systems of the present invention and can thus be incorporated into the methods provided herein.

In some embodiments of any of the preceding methods, the measure of risk is further based on one or more language characteristics derived from a language sample. The language sample may be elicited by a digital prompt, a questionnaire, or a clinician administered interview. In some embodiments, the language sample is, or may be derived from, a dream report, a description of a picture, a thematic apperception test, or a neutral text reading. In some embodiments, the language sample is obtained by passive acquisition (e.g., constant or arbitrary monitoring of outgoing audio data or text data). In some embodiments, the language sample is a text sample and/or an audio sample. In some embodiments, the audio sample is analyzed to derive the one or more language characteristics, wherein the one or more language characteristics comprises one or more acoustic features (e.g., a measure of irregular pitch (e.g., standard variance of pitch), zero-crossing rate, kurtosis energy, HNR, mel-frequency cepstral coefficients MFCC, and frame energy). In some embodiments, the audio sample is transcribed into text.

In some embodiments, the language sample is analyzed to derive the one or more language characteristics, wherein the one or more language characteristics comprises a measure of semantic coherence. A low measure of semantic coherence may be positively correlated with the risk of developing psychosis. In some embodiments, the one or more language characters include a measure of syntactic complexity. A low measure of syntactic complexity may be positively correlated with the risk of developing psychosis. In some embodiments, the one or more language characters include a measure of maximum phrase length. A low measure of maximum phrase length may be positively correlated with the risk of developing psychosis. In some embodiments, the one or more language characters include a measure of lexicon breadth and/or lexicon depth. A low measure of lexicon breadth or depth may be positively correlated with the risk of developing psychosis. In some embodiments, the one or more language characters include a measure of logorrhea. A high measure of logorrhea may be positively correlated with the risk of developing hypomania or mania. In some embodiments, the one or more language characters include a measure of psychometrics (e.g., latent inhibition). In some embodiments, the one or more language characters include a measure of flight of thought. A high measure of flight of thought may be positively correlated with the risk of developing hypomania or mania. In some embodiments, the one or more language characters include a measure of recursiveness. A high measure of recursiveness may be positively correlated with the risk of developing hypomania or mania.

In any of the preceding aspects, the measure of risk may be further based on a result of an EMA. In some embodiments, the measure of risk refers to a risk or precipitating or exacerbating hypomania or mania, and the EMA is a psychopathology questionnaire configured to assess hypomania or mania. In such instances, the EMA can be the Hypomania/Mania Symptom Checklist (HCL-32), the Clinician-Administered Rating Scale for Mania (CARS-M), the Young Mania Rating Scale (YMRS), or an equivalent variant thereof. In other embodiments, the measure of risk refers to a risk of precipitating or exacerbating psychosis, and the EMA is a psychopathology questionnaire configured to assess psychosis (e.g., the psychosis screening questionnaire, the Schizophrenia Test and Early Psychosis Indicator (STEPI), the Cognitive Biases Questionnaire for psychosis (CBQp), or an equivalent variant thereof).

In any of the preceding aspects, the measure of risk can be determined using a machine learning algorithm. In some embodiments, the measure of risk is determined using a cluster model (e.g., a supervised cluster model or an unsupervised cluster model). The measure of risk may be determined using a Random Forest classifier or a within-patient Naïve Bayes classifier. In some embodiments, the measure of risk is determined based on a change of one or more of the characteristics relative to a reference characteristic (e.g., a subject's baseline measurement of the characteristic obtained from the patient at an earlier time point or a cumulative value derived from a plurality of individuals (e.g., healthy individuals)). In some embodiments, the reference characteristic is a predetermined threshold.

In some embodiments of any of the preceding aspects, the psychedelic therapy is being administered for treatment of condition (e.g., a chronic condition). In some embodiments, the condition is an inflammatory-related condition. In some embodiments, the condition is Alzheimer's disease. In some embodiments, the condition is depression (e.g., major depression, melancholic depression, atypical depression, or dysthymia). In some embodiments, the condition is a psychological disorder selected from the group consisting of an anxiety disorder, an addiction, a compulsive behavior disorder, or a symptom thereof. In some embodiments, the psychedelic agent is being administered for improvement of mood or enhancement of performance. In some embodiments, the psychedelic agent is being administered for treatment of stress, treatment of anxiety, treatment of addiction, treatment of depression, or treating of a compulsive behavior. In some embodiments, the psychedelic therapy is being administered for treatment to improve the mental well-being of a patient. In some embodiments, the psychedelic therapy is being administered to reduce the risk of occurrence or reoccurrence of a psychopathology.

In some embodiments, the psychedelic therapy is part of a complex therapy, wherein the patient is additionally being treated with a psychotherapy. In some embodiments, the psychotherapy comprises behavioral activation therapy, talk therapy, existential therapy, and/or self-actualization therapy. For example, the behavioral activation therapy can be brief behavioral activation for depression (BATD). In some embodiments, the complex therapy is provided to the patient in a specialized treatment facility.

In some embodiments of any of methods described herein, the candidate or patient has a neurodegenerative disease (e.g., Alzheimer's disease). In such embodiments, the methods of the invention can be performed after the patient has undergone one or more cognitive assessments. Alternatively, the method includes conducting one or more cognitive assessments on the patient. In some embodiments, the treatment is discontinued based on a negative result of the cognitive assessment (i.e., a result associated with drug-related brain decline). In some embodiments, the cognitive assessment is a mini-mental state examination (MMSE), the Montreal cognitive assessment (MOCA), or the Alzheimer's disease assessment scale—cognitive subscale (ADAS-Cog). In some embodiments, the method includes discontinuing treatment based on behavioral characteristic derived from an interaction between the patient and a device, e.g., using the methods described in U.S. Pat. No. 9,474,481, which is incorporated herein by reference in its entirety.

In some embodiments of any of the preceding aspects, the psychedelic therapy includes administration of an agent selected from the group consisting of a 5-HT_{2A} receptor agonist, an empathogenic agent, and a dissociative agent. For example, in some embodiments, the psychedelic therapy includes administration a 5-HT_{2A} receptor agonist selected from the group consisting of lysergic acid diethylamide (LSD), psilocybin, DOI (±)-1-(2,5-dimethoxyphenyl)-2-aminopropane hydrochloride; (R)-DOI ((R)-1-(2,5-dimethoxy-4-iodophenyl)-2-aminopropane); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl) methylamine) ayahuasca; 3,4,5-trimethoxyphenethylamine (mescaline); 5-methoxy-N,N-dimethyltryptamine (5-meo-DMT); ibogaine; a compound of

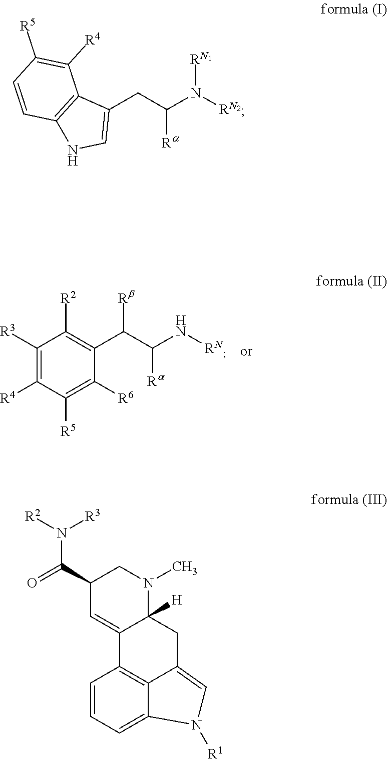

wherein R^{α}, R^{N}₁, R^{N}₂, R⁴, and/or R⁵ of formula (I) are selected from the group consisting of C, CH₃, OH, F, OCH₃, and H; R^{α}, R^{β}, R², R³, R⁴, R⁵, R⁶, and/or R^{N} of formula (II) are selected from the group consisting of OCH₃, CH₃, SCH₃, Br, I, CH₂CH(CH₃)₂, and H; or R¹, R², and/or R³ of formula (III) are selected from the group consisting of CH₂CH₃, CH(CH₃)CH₂CH₃, CH(CH₃)CH₂CH₂CH₃, C₂H₅, CH₂CH₂CH₃, CH(CH₃)₂, and H; or a pharmaceutically acceptable salt thereof.

In some embodiments, the psychedelic agent is an empathogenic agent (e.g., MDMA). In some embodiments, the psychedelic agent is a dissociative agent (e.g., ketamine or esketamine).

In some embodiments of any of the preceding methods, the invention features a software program configured for assessing a risk of precipitating or exacerbating psychosis, hypomania, or mania in a patient undergoing treatment with a psychedelic agent or a candidate for treatment with a psychedelic agent, wherein the software program features computer-readable instructions for performing the method of any of the preceding aspects.

In another aspect, the invention features a software program configured for assessing a risk of precipitating or exacerbating psychosis, hypomania, or mania in a patient undergoing treatment with a psychedelic agent or a candidate for treatment with a psychedelic agent, the software program comprising computer-readable instructions for: (i) obtaining one or more language and/or behavioral samples from the user; (ii) deriving one or more language characteristics from the one or more language samples and/or one or more behavioral characteristics from the one or more behavioral samples; and based on the one or more language and/or behavioral characteristics, determining a measure of risk, wherein the measure of risk correlates with a risk of precipitating or exacerbating psychosis, hypomania, or mania in the candidate; and (iii) reporting the measure of risk to the user and/or a third party.

In some embodiments, the software program further includes computer-readable instructions for receiving information regarding the treatment with the psychedelic agent, wherein the information is selected from the group consisting of psychedelic agent composition, a quantity of psychedelic agent prescribed, a dosing schedule, a quantity of psychedelic agent administered per dose, a frequency of doses administered, and a cumulative quantity of psychedelic agent administered. The computer-readable instructions for receiving information regarding the treatment with the psychedelic agent may be configured to receive the information from the patient, a clinician, or the third party. In some embodiments, the computer-readable instructions for receiving information regarding the treatment with the psychedelic agent are further configured to store and/or report the information regarding the treatment with the psychedelic agent (e.g., all or a portion of the information can be reported to the patient or the third party, e.g., all or a portion of the information can be reported to a clinician or another third party upon detecting non-compliance by the patient, e.g., an increase or decrease in the dose or frequency of psychedelic agent administered).

In some aspects, the invention features a computer system for assessing a risk of precipitating or exacerbating psychosis, hypomania, or mania in a patient undergoing treatment with a psychedelic agent or a candidate for treatment with a psychedelic agent, the computer system configured to run the any software program of the present invention.

In another aspect, the invention features a computer system for assessing a risk of precipitating or exacerbating psychosis, hypomania, or mania in a patient undergoing treatment with a psychedelic agent or a candidate for treatment with a psychedelic agent, the computer system including: (i) a mobile device comprising one or more input mechanisms, a processor, and one or more output mechanisms; and (ii) a software program readable by the processor, the software program featuring instructions for: (a) using the one or more input mechanisms, obtaining one or more language and/or behavioral samples from the user; (b) using the processor, deriving one or more language characteristics from the one or more language samples and/or one or more behavioral characteristics from the one or more behavioral samples; and based on the one or more language and/or behavioral characteristics, determining a measure of risk, wherein the measure of risk correlates with a risk of precipitating or exacerbating psychosis, hypomania, or mania in the candidate; and (c) using the one or more output mechanisms, reporting the measure of risk to the user and/or a third party.

In some embodiments, the computer system includes a software program that further includes computer-readable instructions for receiving information regarding the treatment with the psychedelic agent, wherein the information is selected from the group consisting of psychedelic agent composition, a quantity of psychedelic agent prescribed, a dosing schedule, a quantity of psychedelic agent administered per dose, a frequency of doses administered, and a cumulative quantity of psychedelic agent administered. The computer-readable instructions for receiving information regarding the treatment with the psychedelic agent may be configured to receive the information from the patient, a clinician, or the third party. In some embodiments, the computer-readable instructions for receiving information regarding the treatment with the psychedelic agent are further configured to store and/or report the information regarding the treatment with the psychedelic agent (e.g., all or a portion of the information can be reported to the patient or the third party, e.g., all or a portion of the information can be reported to a clinician or another third party upon detecting non-compliance by the patient, e.g., an increase or decrease in the dose or frequency of psychedelic agent administered).

In some embodiments of any of the software programs of computer systems described above, the software program further includes a psychotherapy application, wherein the psychotherapy application is configured to provide psychotherapy to the patient or candidate. For example, the psychotherapy can be provided via telemedicine. In some embodiments, the psychotherapy is behavioral activation therapy.

As used herein, “well-being” refers to a positive state of health or comfort, e.g., relative to a reference population. As used herein “mental well-being” refers to a positive mental state, relative to a reference population. For example, in an individual having depression, low self-esteem, addiction, compulsion, or anxiety may experience an improvement in mental well-being in response to therapy aimed at improving mood, self-esteem, addiction, compulsion, or anxiety, respectively. As used herein, “physical well-being” refers to one or more positive aspects of an individual's physical health. For example, an improvement of physical well-being includes alleviation of somatic symptoms associated with a psychological disorder, depression, addiction, compulsion, anxiety, or sexual dysfunction. Such symptoms include, for example, chronic pain, pain disorder, relational disorder, body dysmorphia, conversion (e.g., loss of bodily function due to anxiety), hysteria, neurological conditions without identifiable cause, or psychosomatic illness).

As used herein, a “psychological disorder” refers to a condition characterized by a disturbance in one's emotional or behavioral regulation that reflects a dysfunction in the psychological, biological, or developmental processes underlying mental function. Psychological disorders include, but are not limited to depressive disorders (major depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcohol, tobacco, or drug abuse)), and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms. Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, Va.; American Psychiatric Association, 2013).

As used herein, a “psychedelic agent” refers to a compound capable of inducing an altered state of consciousness, i.e., a marked deviation in the subjective experience or psychological functioning of a normal individual from his or her usual waking consciousness. Altered states of consciousness can be monitored, evaluated, and/or quantified using any of a variety of methods known in the art including, without limitation, Dittrich's APZ (Abnormal Mental States) questionnaire, and its revised versions, OAV and 5D-ASC (see, for example, Dittrich et al., A Pharmacopsychiatry 1998, 31:80; Studerus et al., PLoS ONE 2010, 5). Psychedelic agents include 5-HT_{2A} agonists (e.g., lysergic acid diethylamide (LSD), empathogenic agents (i.e., serotonin (5-HT) releasing agents; e.g., MDMA), and dissociative agents (i.e., N-Methyl-D-aspartate (NMDA) receptor agonists; e.g., ketamine).

As used herein, a “5-HT_{2A} agonist” refers to a compound that increases the activity of a 5-hydroxytryptamine 2A receptor. Examples of such agonists include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); ibogaine; a compound of formula (I), wherein R^{α}, R^{N}₁, R^{N}₂, R⁴, and/or R⁵ are selected from the group consisting of C, CH₃, OH, F, OCH₃, and H; a compound of formula (II), wherein R^{α}, R^{β}, R², R³, R⁴, R⁵, R⁶, and/or R^{N} are selected from the group consisting of OCH₃, CH₃, SCH₃, Br, I, CH₂CH(CH₃)₂, and H; or a compound of formula (III), wherein R¹, R², and/or R³ are selected from the group consisting of CH₂CH₃, CH(CH₃)CH₂CH₃, CH(CH₃)CH₂CH₂CH₃, C₂H₅, CH₂CH₂CH₃, CH(CH₃)₂, and H. Compounds of formulas (I)-(III) can be synthesized using methods known in the art, including, for example, procedures described in Kornfeld et al. (J. Am. Chem. Soc. 1954, 76(20):5256-5257), Kornfeld et al. (J. Am. Chem. Soc. 1956, 78(13):3087-3114), Marino et al. (J. Org. Chem. 1995, 60(9):2704-2713), and Tsao (J. Am. Chem. Soc. 1951, 73(11):5495-5496).

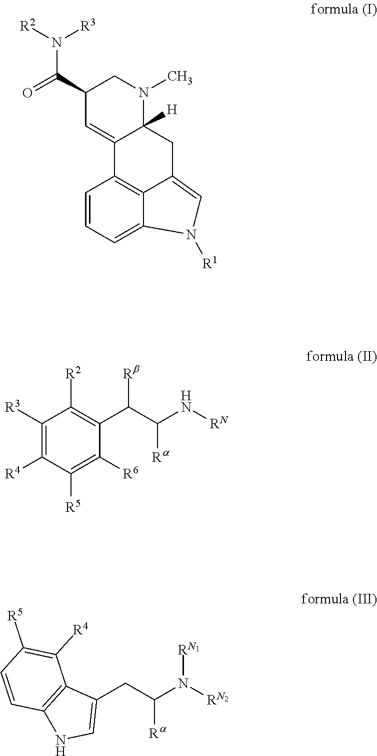

As used herein, the term “treating” refers to administering a pharmaceutical composition for therapeutic purposes. To “treat a disorder” or use for “therapeutic treatment” refers to administering treatment to a patient already suffering from a disease to ameliorate the disease or one or more symptoms thereof to improve the patient's condition. The methods of the invention can also be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

The term “administration” or “administering” refers to a method of giving a dosage of a pharmaceutical composition to a subject, where the method is, e.g., oral, topical, transdermal, by inhalation, intravenous, intraperitoneal, intracerebroventricular, intrathecal, or intramuscular.

As used herein, a “psychotherapy” refers to a non-pharmaceutical therapy in which the subject is psychologically engaged, directly or indirectly (e.g., by dialogue), in an effort to restore a normal psychological condition; to reduce the risk of developing a psychological condition, disorder, or one or more symptoms thereof; and/or to alleviate a psychological condition, disorder, or one or more symptoms thereof. Psychotherapy includes Behavioral Activation (BA), Cognitive Behavioral Therapy (CBT), Interpersonal psychotherapy (IPT), Psychoanalysis, Hypnotherapy, Psychedelic Psychotherapy, Psycholytic Psychotherapy, and other therapies. In some embodiments, a subject undergoes psychotherapy in conjunction with (e.g., prior to, during, and/or after) a pharmaceutical therapy, such as a psychedelic therapy.

As used herein, a “psychotherapeutic setting” refers to an environment configured to be substantially free of distraction or stress to facilitate a positive therapeutic response in a subject.

As used herein, a “specialized treatment facility” refers to a particular psychotherapeutic setting in which subjects are evaluated for treatment by a complex therapy (i.e., a therapy including both pharmaceutical (e.g., psychedelic) and non-pharmaceutical (e.g., psychotherapy) treatments).

Other features and advantages of the invention will be apparent from the following Detailed Description, Examples, Figure, and Claims.

### BRIEF DESCRIPTION OF THE DRAWING

The following drawing is illustrative of a particular embodiment of the present invention and is not limiting to various embodiments encompassed by the present invention.

FIG. 1 is a flow chart illustrating various exemplary behavioral and language sample inputs, processing steps, and output of an exemplary computer system provided by the present invention.

### DETAILED DESCRIPTION

The invention features a method of enhancing the safety of the therapeutic application of psychedelic, empathogenic, and dissociative compounds including: evaluating the suitability of the treatment for the patient given the capacity to detect a latent, remitted, or active psychosis, hypomania, or mania. The invention includes a software application delivered via computer, smartphone, or other device (e.g. mobile device), which is capable of collecting patient data through textual and/or audio recording of responses to automated and clinician administered structured interviews and surveys, audio recording of phone conversations, mobile sensors and other psychometric information gathered from a smartphone or other mobile device, and other prompted and unprompted voice, text, keypad, push-button, or other forms of computerized data captures. The invention is further capable of converting this data into a format capable of being rapidly analyzed in automated assessments in one or more validated quantitative frameworks capable of identifying prodromal or manifest symptoms of psychosis, hypomania, and mania. The invention is also capable of recording the results of these automated quantitative assessments and making them available to supervisory clinicians should they indicate a patient is in need of immediate attention. The invention is also capable of alerting clinicians should these results indicate a patient is at risk of developing psychosis, hypomania, or mania, or is currently experiencing a psychotic or manic condition, enabling a retest of the patient to avoid possible false-positive result, excluding the patient if they have not yet commenced a drug treatment, and if warranted, emergency medical intervention and/or discontinuation of the associated drug treatment. In addition, methods of the invention can trigger clinical assessment (e.g., by a clinician or by software) for further review/confirmation and to determine the need to adjust a therapy (e.g., a chronic, acute, or psychotherapy-assisted dosing regimen). For example, a therapy may be adjusted by supplementing the therapy, escalating the dose, reducing the dose, retreating the patient, discontinuing treatment, or otherwise modifying a prescribed course of therapy. A clinical assessment may also trigger contacting a patient's care provider, family, next of kin, etc.

An evaluation can take place prior to starting a regimen of psychedelic therapy, e.g., in which an individual has otherwise been categorized as a candidate for psychedelic therapy for treatment of a particular indication. The evaluation provides an indication of the candidate's likelihood of developing psychosis, hypomania, or mania (e.g., according to a predisposition, e.g., a genetic predisposition), which may be exacerbated or triggered by a psychedelic therapy. Accordingly, in one embodiment of the invention, a predisposition toward developing psychosis, hypomania, or mania (e.g., as indicated by a measure of risk provided by the evaluation) suggests that a candidate should not be treated using a psychedelic treatment, and an alternate course of treatment may be indicated (e.g., psychotherapy and/or an alternative pharmaceutical regimen). Alternatively, a low risk of developing psychosis, hypomania, or mania, as indicated by the screening methods provided herein, can indicate that a candidate is fit for a regimen of psychedelic therapy (e.g., as part of a complex therapy, e.g., including a psychotherapy).

Alternatively, the evaluation can occur at one or more time points within an ongoing psychedelic therapy (e.g., within or between regimens). For example, a patient being treated using psychedelic therapy can be screened to monitor any development or emergence of a risk of prodromal or symptomatic psychosis, hypomania, or mania corresponding with treatment with a psychedelic agent (e.g., as a cause of the psychedelic agent or correlated with treatment with the psychedelic agent). Accordingly, the methods of screening provided herein may inform a determination that a patient undergoing psychedelic therapy is at risk of developing psychosis, hypomania, or mania (e.g., an elevated risk prior to an earlier time point, which may be indicative of pre-symptomatic development of psychosis, hypomania, or mania), in which instance a decision or recommendation can be made regarding continued therapy (e.g., whether to adjust dosage or suspend administration of the psychedelic therapy). Alternatively, a patient who is undergoing a low dose regimen of psychedelic therapy who displays no risk of developing psychosis, hypomania, or mania using the methods and systems of the invention may be determined to qualify for a higher dose.

### Assessing Risk of Developing Psychosis, Hypomania, or Mania

Psychedelic therapies can be safely administered if a candidate is identified as having a low risk of developing psychosis, hypomania, or mania. Similarly, a patient undergoing psychedelic therapy can be monitored to ensure that a risk of developing psychosis, hypomania, or mania does not increase during the course of treatment. Thus, the methods and systems described herein involve screening of patients and candidates to determine a measure of risk correlating with the likelihood of developing psychosis, hypomania, or mania.

Risk factors for development of psychosis, hypomania, or mania can be derived from various characteristics (e.g., behavioral characteristics and/or verbal and non-verbal language characteristics). For example, a reduction in sematic coherence can be associated with prodromal or symptomatic psychosis, hypomania, or mania. Similarly, changes in social behavior patterns can be associated with hypomania or mania. Often, such changes in behavior or language are subtle and/or unobservable for consistent periods, and qualitative characterization thereof is inaccurate. Provided herein are methods and systems that automate detection of such risk factors to enhance sensitivity of particular behavioral and language characteristics and integrate multiple characteristics to enhance the accuracy of risk prediction and clinical safety.

### Language Sample Acquisition

A language sample can be an audio sample (e.g., a verbal sample) and/or text sample (e.g., a typed or transcribed sample (e.g., a transcription from a verbal sample)). Language samples can be passively acquired (e.g., recorded, e.g., from conversations that the candidate or patient has using their mobile device). Additionally or alternatively, language samples can be actively obtained. For example, language samples can be elicited by a digital prompt, such as a written question or statement on a computer or mobile device interface, to which the candidate or patient may respond (e.g., verbally or by typing). A digital prompt may include one or more prerecorded audio or audiovisual questions or statements to which the candidate or patient responds (e.g., verbally or by typing). Accordingly, systems of the invention include a detection means, such as a microphone, keypad, or digital touchscreen to accept a language sample input from a candidate or subject. In some embodiments, the systems described herein include a feature that records only outgoing speech from a smartphone.

In some embodiments, a clinician elicits the language sample from the candidate or patient, e.g., by administering an interview or survey. In this instance, a clinician may be in the same room as the candidate or patient, or the clinician may elicit the language sample from a remote location, e.g., via telemedicine, e.g., over a telephone or internet connection.

Whether the language sample is elicited by a preset digital prompt, by a face-to-face interaction, or via telemedicine, it may feature one or more open ended questions or commands, e.g., as an open ended interview, to elicit free speech, which has been shown to provide a suitable source for predictive language characteristics. Exemplary methods thereof are described, for example, by Bedi et al (npj Schizophrenia 2015, 1:15030), incorporated herein by reference in its entirety.

In some embodiments, active acquisition of a language sample (e.g., an audio sample) involves eliciting speech, for example, as part of a dream report, a description of an image (e.g., as part of a thematic apperception test), or as a neutral text reading.

In other embodiments, acquisition of a language sample (e.g., an audio sample) is a passive acquisition. A passive acquisition of a language sample includes recordings of telephone conversations, such as described in Muaremi (Muaremi et al., (eds) MindCare 2014. Lecture Notes of the Institute for Computer Science, Social Informatics and Telecommunications Engineering, 100. Springer, Cham).

A language sample may be of any size or length suitable to provide one or more speech characteristics. For example, an audio language sample (e.g., an audio recording) can be from 1 millisecond to 120 minutes in length. In general, an acoustic analysis requires a shorter audio sample than a semantic analysis. In some embodiments, a sample useful for acoustic analysis is from 1 millisecond to 120 minutes in length (e.g., from 1 millisecond to 10 milliseconds, from 10 milliseconds to 20 milliseconds, from 20 milliseconds to 30 milliseconds, from 30 milliseconds to 40 milliseconds, from 40 milliseconds to 50 milliseconds, from 50 milliseconds to 100 milliseconds, from 100 milliseconds to 500 milliseconds, from 500 milliseconds to 1 second, from 1 second to 10 seconds, from 10 seconds to 30 seconds, from 30 seconds to 1 minute, from 1 minute to 10 minutes, from 10 minutes to 30 minutes, or from 30 minutes to 120 minutes in length).

In some embodiments, a sample useful for semantic analysis is from 5 seconds to 120 minutes in length (e.g., from 10 seconds to 90 minutes, from 20 seconds to 60 minutes, from 30 seconds to 45 minutes, from 1 minute to 30 minutes, or from 5 minutes to 15 minutes in length; e.g., from 10 seconds to 20 seconds, from 20 seconds to 30 seconds, from 30 seconds to 1 minute, from 1 minute to 5 minutes, from 5 minutes to 10 minutes, from 10 minutes to 20 minutes, from 20 minutes to 30 minutes, from 30 minutes to 60 minutes, from 60 minutes to 90 minutes, or from 90 minutes to 120 minutes in length). In some embodiments, an audio language sample (e.g., an audio recording) can be less than 60 minutes (e.g., less than 50 minutes, less than 45 minutes, less than 40 minutes, less than 35 minutes, less than 30 minutes, less than 25 minutes, less than 20 minutes, less than 15 minutes, less than 10 minutes, less than 5 minutes, less than 4 minutes, less than 3 minutes, less than 2 minutes, less than 60 seconds, less than 50 seconds, less than 40 seconds, less than 30 seconds, less than 20 seconds, or less than 10 seconds).

In some embodiments, a language sample (e.g., an audio sample (e.g., a verbal sample) and/or text sample (e.g., a typed or transcribed sample (e.g., a transcription from a verbal sample)) comprises from 20 words to 10,000 words (e.g., from 30 words to 8,000 words, from 50 words to 5,000 words, from 100 words to 2,500 words, or from 500 to 1,000 words; e.g., from 20 words to 30 words, from 30 words to 40 words, from 40 words to 50 words, from 50 words to 75 words, from 75 words to 100 words, from 100 words to 200 words, from 200 words to 500 words, from 500 words to 1,000 words, from 1,000 words to 2,500 words, from 2,500 words to 5,000 words, from 5,000 words to 7,500 words, or from 7,500 words to 10,000 words). In some embodiments, a language sample comprises less than 10,000 words (e.g., less than 9,000 words, less than 8,000 words, less than 7,000 words, less than 6,000 words, less than 5,000 words, less than 4,000 words, less than 3,000 words, less than 2,000 words, less than 1,000 words, less than 900 words, less than 800 words, less than 700 words, less than 600 words, less than 500 words, less than 400 words, less than 300 words, less than 200 words, less than 100 words, less than 90 words, less than 80 words, less than 70 words, less than 60 words, or less than 50 words).

In some embodiments, the language sample is processed in real time, and the software continues to record conversations or the prompt continues to elicit a language sample for the duration of time necessary to achieve a significant result. For example, the system may iteratively analyze the accumulating language sample until one or more characteristics or risk measures reaches a predetermined significance level, at which point the prompt may automatically conclude.

### Language Analysis

The methods and systems provided herein feature automated language analysis to process and analyze one or more language samples obtained from a candidate or patient. Automated language analysis can be executed as part of a software program (e.g., as part of a software application or accessible to the software application (e.g., on a remote server in communication with the software application)).

In some embodiments, the invention provides methods and systems that utilize automated speech analysis to determine a risk measure. An exemplary automated speech analysis procedure that can predict onset of psychosis is described by Bedi (Bedi et al., npj Schizophrenia 2015, 1:15030), which is incorporated herein by reference in its entirety.

In another embodiment, the invention provides methods and systems that utilize automated acoustic analysis to determine a risk measure. An exemplary automated acoustic analysis procedure that can predict onset of psychosis is described by Faurholt-Jepsen (Faurholt-Jepsen et al., Transl Psychiatry 2016, 6:e856), which is incorporated herein by reference in its entirety.

In general, language analysis begins with an input of a language sample (e.g., an audio sample (e.g., a verbal sample) and/or text sample (e.g., a typed or transcribed sample (e.g., a transcription from a verbal sample)). Pre-processing steps may be employed, such as transcribing verbal speech from an audio file into text. Additional pre-processing steps can be performed as described by Bedi, for example, using available tools, such as the Natural Language Toolkit (http://www.nitk.org). For example, punctuation may be removed, and phrases may be parsed. In some instances, words can be converted to the roots from which they are inflected, or lemmatized, e.g., using NLTK Wordnet lemmatizer. In some embodiments of the methods provided herein, pre-processed data includes a list of words that have been lemmatized, parsed into phrases, converted to lower case, maintained in their original order, and/or punctuation removed. Voice features can be extracted as described in Faurholt-Jepsen, for example, using available tools, such as openSMILE toolkit (Eyben F, et al., Proceedings of ACM Multimedia: Firenxe, Italy, 2010).

After any necessary pre-processing steps are performed, one or more characteristics of the language sample can be derived. Characteristics that can be derived from a language sample (e.g., a typed or transcribed sample (e.g., a transcription from a verbal sample)) include semantic coherence, syntactic complexity, comprehension, lexicon depth, lexicon breadth, or semantic proximity to one or more concepts related to an influence of a psychedelic agent (e.g., as described in the 5D-ASC rating scale). In general, a low measure of semantic coherence (e.g., similarity among pairs of consecutive phrases, or pairs of phrases separated by an intervening phrase) is positively correlated with risk of developing psychosis. Similarly, a low measure of syntactic complexity may be positively correlated with risk of developing psychosis; a low measure of lexicon depth may be positively correlated with risk of developing psychosis; and/or a low measure of lexicon breadth may be positively correlated with risk of developing psychosis. Less verbosity (e.g., a maximum number of words per phrase) can also be indicative of development of psychosis.

Language characteristics that can correlate with hypomania or mania include logorrhea (e.g., excess wordiness, higher Type/Token ratio, or incoherence), and recursiveness (e.g., returns to the same topic). Thus, in some instances, a measure of logorrhea and/or recursiveness can be used to derive a measure of risk of exacerbating or precipitating hypomania or mania.

As a derivation of any of the characteristics described herein is unique to its experimental circumstances (i.e., it is qualitative and relative), any of the preceding methods of deriving a characteristic from a language sample may result in a “measure” of that characteristic, which herein encompasses any value into which the quantity of the characteristic factors. For example, in some embodiments, semantic coherence may be weighted to a greater extent than syntactic complexity in an algorithm for calculating risk of psychosis.

Audio samples may provide further language characteristics from which a measure of risk of psychosis, hypomania, or mania can be derived. For example, speech quantity, rate, and fluctuation in pitch can be correlated with a high risk of developing hypomania or mania.

Acoustic features, such as pitch frequency F0, zero-crossing rate (ZCR), harmonics to noise ratio (HNR), mel-frequency cepstral coefficients (MFCC), and root mean squared (RMS) frame energy can be associated with prodromal psychosis, hypomania, or mania. Other acoustic features and their predictive value of psychosis, hypomania, and/or mania known in the art are contemplated for use as part of the methods and systems described herein. Such acoustic features can be characterized or summarized using known methods, including spectral mapping and statistical functions, such as means, standard deviations, ranges, kurtosis energies, extremes, moments, segments, peaks, linear and quadratic regressions and coefficients thereof, percentiles, durations, onsets, zero-crossings, modulation spectra, and composites thereof.

In some embodiments, the methods and systems of the invention feature a toolkit (e.g., openSMILES), which can be run directly, for example, on a mobile device (e.g., a smartphone or tablet) in real time, as the audio sample is acquired. In some instances, raw or processed data can be input into a model that is user-specific (e.g., compared with baseline values of the same user) or user-independent (e.g., compared with a predetermined threshold or a composite of values obtained by other users). Additional methods of measuring, processing, and characterizing any of the aforementioned acoustic features are known in the art and describe, for example, in Vanello et al., Conf Proc IEEE Eng Med Biol Soc 2012. 2012:2104-2107 and Karam et al., Proceedings of the IEEE International Conference on Acoustics, Speech, and Signal Processing (Conference) 2014:4858-4862, both of which are incorporated by reference in their entireties.

Methods of deriving characteristics from language samples are known in the art. For example, latent semantic analysis (LSA) can be used to analyze language samples to derive characteristics involved in psychosis, as described in Landauer et al. (Psychol Rev 1997, 104:211-240) and Bedi, both of which are incorporated by reference. Alternative methods for deriving characteristics correlating with risk of developing psychosis, hypomania, or mania are described, for example, in Bedi et al. (Neuropsychopharmacology 2014, 39, 2340-2348) and Mota et al. (Scientific Reports 2014, 3691).

In some embodiments of any of the methods described above, a measure of any characteristic of a language sample can be derived using a machine learning algorithm, as described, for example, in Bedi. In some embodiments, a supervised or unsupervised cluster model can be used, e.g., to classify characteristics among a population of candidates or patients, or to compare one or more characteristics of a candidate or patient with a those of a reference population. The measure of risk may be determined using a Random Forest classifier or a within-patient Naïve Bayes classifier.

A measure of risk of developing psychosis, hypomania, or mania can be determined using one or more characteristics of a language sample obtained using any of the methods described herein. As part of this step, in some embodiments of the methods and systems disclosed herein, a machine learning algorithm can be utilized. Similarly, a supervised or unsupervised cluster model can be used, e.g., to classify a measure of risk of a candidate or patient, e.g., as high risk or low risk.

In any of the preceding methods of language analysis, a measure of a characteristic can be determined based on comparison with a reference characteristic (i.e., a characteristic derived from one or more individuals of a reference population). In any of the methods disclosed herein, a change in a language characteristic may be identified by comparison with one or more prior language samples, such as a comparison to a baseline measure (e.g., at a time point in which the candidate or patient did not have prodromal or symptomatic psychosis, mania, or hypomania).

In some embodiments, a measure of risk is determined by a composite score of two or more characteristics of a language sample (e.g., a text sample and/or an audio sample). For example, a measure of risk can be determined by considering one, two, three, four, five, or six of any of the characteristics selected from the group consisting of semantic coherence, syntactic complexity, comprehension, lexicon depth, or lexicon breadth.

### Behavioral Sample Acquisition

Various behavioral characteristics can be derived from one or more behavioral samples. In some instances, a behavioral sample is a measure of social behavior and/or physical activity (e.g., mobility, physiology, or other actions (e.g., psychomotor activity)). In many instances, a behavioral sample is passively acquired. In such instances, systems of the invention may feature acquisition programs that constantly or intermittently acquire data. Passive acquisition can occur at predetermined time points and/or for predetermined durations. In other instances, passive acquisition of behavioral samples can be initiated manually, e.g., by a third party, e.g., by a clinical professional. Initiation of passive acquisition may, for example, occur in response to a change in one or more characteristics being monitored, such as an increase one or more characteristics factoring into a measure of risk.

In some embodiments, a behavioral sample is a telephone record, which contains information related to an individual's social behavior (e.g., a number or frequency of outgoing calls or messages, a number or frequency of incoming calls or messages, a ratio between a number or frequency of outgoing calls or messages and a number or frequency of incoming calls or messages, a duration of one or more calls, a length of one or more messages, a number or frequency of newly added contacts, a number of changes of turns between participants of a telephone call, a number of short turns in conversation, a number of changes in cell tower IDs, and/or a number of unique cell tower IDs). Systems of the invention may be configured to automatically receive an individual's telephone record upon its preparation (e.g., daily, weekly, biweekly, or monthly) or in real time.

A behavioral sample may also be a report of the number and/or frequency of instances in which a mobile device screen (e.g., a screen of a smartphone or tablet) is turned on. Such a sample can be detected by a program running on device being monitored, or the device screen status can be detected remotely.

In other instances, a behavioral sample detected by a sensor. For example, physical activity of an individual may be detected or monitored by a sensor. For example, physical sensors include any device able to detect physical activity or characteristics (e.g., mobility, physiology, and/or motion, e.g., psychomotor activity), including video sensors (e.g., video cameras), motion sensors (e.g., passive infrared sensors, ultrasonic sensors, microwave sensors, or tomographic sensors), GPS, accelerometers (e.g., as part of a mobile device, such as a smartphone or smart wearable device), or biosensors (e.g., sensors that detect physiological characteristics, such as body mass, body temperature, heart rate, breathing characteristics (e.g., rate or depth), or blood characteristics (e.g., blood pressure, blood glucose levels, blood-drug concentration (e.g., blood-alcohol concentration))).

Biosensors may be part of a mobile device, such as a smartphone, tablet, or wearable mobile device, such as a watch, bracelet, or necklace. Biosensors include sensors equipped with the capacity to detect the presence or level of one or more biomarkers (e.g., digital biomarkers), such as CO₂ levels (e.g., blood CO₂), glucose levels, expression of genes or proteins that correlate positively or negative with behavior.

Sensors, such as biosensors, can also detect changes in sleep behavior. Alternatively, changes in sleep behavior can be detected and monitored using sensors that detect levels of light exposure. For example, high relative light exposure can indicate that the candidate or patient is sleeping less, and vice-versa. Characteristics associated with abnormal sleep patterns can be identified using the present methods, for example, as described in Kumar, et al., Value in Health 2017, 20(5): A54. For example, sleep deprivation can correlate with depression.

In addition, speed of typing can be utilized as a behavioral characteristic. Fast or slow typing can be correlated with various characteristics, such as cognitive function, as described in Dagum, npj Digital Medicine 2018, 1(10): 58-70, which is incorporated herein by reference in its entirety. Physical activity can also be detected by monitoring signal strength of an individual's mobile device(s) (e.g., a smartphone) relative to a stationary sensor. For example, the frequency and duration of Wi-Fi connections with one or more Wi-Fi networks can be recorded from an individual's mobile device to derive a measure of an individual's mobility. Additionally or alternatively, signal strength (e.g., Wi-Fi signal strength) can be indicative of how much or how far an individual moves around their home. Contact data with cell phone towers and/or other stationary sensors can be analogously utilized to derive a measure of an individual's mobility.

Sensors also include non-physical sensors. For example, systems and methods of the invention may additionally or alternatively access data from programs that track television habits (e.g., frequency of changing channels, genres of programs or movies watched, sound volume, etc.), internet habits (e.g., frequency of opening new webpages, types of sites visited, number of emails sent, frequency of messages sent), music habits (e.g., genre of music listened to, volume of music, frequency of skipped tracks, number of repeated tracks, etc.), and/or eating and/or drinking habits (e.g., as measured by a smart refrigerator). Any known software applications and/or hardware systems capable of tracking such habits are suitable for methods and systems of the present invention.

A behavioral sample may be acquired for any suitable duration to provide one or more behavioral characteristics. In some embodiments, the behavioral sample is processed in real time, and the software continues to acquire the sample for the duration of time necessary to achieve a significant result. For example, the system may iteratively analyze the accumulating language sample until one or more characteristics or risk measures reaches a predetermined significance level, at which point the prompt may automatically conclude.

### Behavioral Analysis

The methods and systems provided herein feature behavioral analysis to process and analyze one or more behavioral samples obtained from a candidate or patient. In some embodiments, behavioral analysis is automated (e.g., as part of a software program or application or accessible to a software program or application (e.g., on a remote server in communication with a software program or application). In some embodiments, methods and systems provided herein include a software program or application featuring behavioral analysis in addition to language analysis, as described above.

In some embodiments, the invention provides methods and systems that utilize automated behavioral analysis to determine a risk measure. Any behavioral samples described above and/or known in the art can be analyzed according to methods described herein or known in the art.

In many instances, disturbances in behavioral rhythms indicate prodromal or symptomatic onset of psychosis, mania, or hypomania. Thus, in any of the methods disclosed herein, a change in a characteristic may be identified by comparing its occurrence with a prior one or more of such occurrences, such as a comparison to a baseline measure (e.g., at a time point in which the candidate or patient did not have prodromal or symptomatic psychosis, mania, or hypomania).

In some embodiments, the invention provides methods and systems that analyze telephone activity of an individual (e.g., activity acquired from a telephone record, as described above). Telephone activity may be, for example, a number or frequency of outgoing calls or text messages, a number or frequency of incoming calls or text messages, a ratio between a number or frequency of outgoing calls or text messages and a number or frequency of incoming calls or text messages, a duration of one or more calls, a length of one or more text messages, a number or frequency of newly added contacts, a number of changes in cell tower IDs, and/or a number of unique cell tower IDs. In some embodiments, a change in telephone activity is an indicator of onset of prodromal or symptomatic psychosis, mania, or hypomania. Correlations between each of the above-referenced characteristics and the risk of developing psychosis, mania, or hypomania are known in the art or provided herein.

For example, in some instances, a number or frequency of outgoing calls or messages is positively correlated with the risk of developing mania or hypomania. In some embodiments, an increase over time in the number of outgoing calls or messages by an individual indicates onset of prodromal or symptomatic psychosis, mania, or hypomania.

A high ratio between the number or frequency of outgoing calls or text messages and a number or frequency of incoming calls or text messages can be indicative of prodromal or symptomatic mania or hypomania. An increase over time in the ratio between the number or frequency of outgoing calls or text messages and a number or frequency of incoming calls or text messages may indicate onset of mania or hypomania.

The duration of telephone calls may positively correlate with prodromal or symptomatic mania or hypomania. In some instances, an increase over time in the duration of telephone calls (e.g., an increase in the average duration of telephone calls or an increase in the number of lengthy telephone calls) indicate onset of mania or hypomania.

In some embodiments, the length of outgoing text messages positively correlates with prodromal or symptomatic mania or hypomania. Thus, an increase over time in the length of outgoing text messages (e.g., an increase in the average length of a text message or an increase in the number of lengthy text messages) can indicate onset of mania or hypomania.

The number or frequency of newly added contacts may positively correlate with prodromal or symptomatic psychosis, mania, or hypomania. In some embodiments, an increase over time in the frequency of newly added contacts indicates onset of mania or hypomania.

Additionally, the number of speaking turns between participants, or the number of short turns in conversation, may correlate with prodromal or symptomatic mania or hypomania.

In some embodiments, the number or frequency of instances in which a mobile device screen is turned on is positively correlated with prodromal or symptomatic psychosis, mania, or hypomania. In some embodiments, an increase over time in the number or frequency of instances in which a mobile device screen is turned on indicates onset of mania or hypomania.

A change in behavioral rhythms can be indicative of onset of prodromal or symptomatic psychosis, mania, or hypomania. Thus, in some embodiments, a change in pattern of any psychomotor activity or mobility described herein (e.g., as monitored or detected using any means described herein or known in the art) indicates onset of mania or hypomania.

A measure of risk of developing psychosis, hypomania, or mania can be determined using one or more characteristics of a behavioral sample obtained using any of the methods described herein. As part of this step, in some embodiments of the methods and systems disclosed herein, a machine learning algorithm can be utilized. Similarly, a supervised or unsupervised cluster model can be used, e.g., to classify a measure of risk of a candidate or patient, e.g., as high risk or low risk.

### Biosensors

In addition to biosensors configured to sense behavioral characteristics, biosensors can be utilized in the methods of the present invention to detect changes in biomarkers indicative of other characteristics. For example, biosensors can be configured to detect presence of a level of a target molecule present in a body sample obtained from the patient (e.g., a level of the psychedelic agent, a metabolite of the psychedelic agent, or another molecule that correlates positively or negatively with the level of the psychedelic agent in the patient). Accordingly, biosensors configured for use in the present methods can allow a clinician or other system (e.g., a software program of the invention) to monitor the amount of a psychedelic agent in a patient at any one or more times, thereby informing decisions regarding dosing (e.g., whether to adjust a dose amount or frequency) and retreatment (e.g., whether a patient should be retreated or the frequency of retreatment). Biomarkers obtained by such biosensors can be referred to as “digital biomarkers.”

Additionally or alternatively, digital biomarkers can inform a measure of risk, such as a risk of having or developing any of the disorders described herein (e.g., paranoid ideation, propensity towards paranoid thinking, paranoid personality disorder, personality disorders, intellectual disabilities (e.g., intellectual developmental disorder), bipolar disorder, depersonalization, dissociation, derealization, hallucinogen-psychoactive abuse, hallucinogen-use disorders, hallucinogen-induced disorders (e.g., hallucinogen-persisting perception disorder (HPPD), or high-risk behavior)). Accordingly, a measure of risk can be derived from any one or more digital biomarkers to inform decisions resulting from the methods of screening and monitoring provided herein.

### Clinical Methods

A candidate or patient can be screened once or on multiple occasions. For example, a candidate or a patient may be screened at least twice (e.g., at least three times, four times, five times, six times, at least seven times, at least eight times, at least nine times, at least ten times, at least twelve times) or more. In some instances, a candidate is screened multiple times prior to determining his or her risk of developing psychosis, hypomania, or mania. In some embodiments, two or more of the multiple screening sessions are different. For example, different characteristics of a behavioral and/or language sample may be derived and/or analyzed for each screening session, or certain features of a behavioral or language sample may be elicited more or less frequently, and/or weighted more or less heavily, over a series of screenings. The difference in characteristics may be dictated according to a differential trend in one feature of a sample versus another, for example, as identified by a machine learning algorithm. For example, a system eliciting both acoustic and text-based samples at an equal frequency may detect an increasing variance in acoustic features without a corresponding change in text-based samples. In response, the system can automatically increase the relative frequency of eliciting acoustic samples to increase sensitivity to the features relevant to a measure of risk.

A patient may be evaluated at regular intervals, e.g., to maximize the chance of detecting emergence of any prodromal or symptomatic psychosis, hypomania, or mania. In some embodiments, the patient is evaluated at least once each year (e.g., once per year, twice per year, three times per year, four times per year, at least five times per year, at least six times per year, at least seven times per year, at least eight times per year, at least nine times per year, at least ten times per year, at least eleven times per year, at least twelve times per year, at least once per three weeks, at least once per two weeks, at least once per week, at least twice per week, at least three times per week, at least four times per week, at least five times per week, at least six times per week, or about once per day).

In some embodiments, a candidate or patient identified as at risk of developing psychosis, hypomania, or mania can undergo additional testing for confirmation or comparison. Any suitable test known in the art may be used. For example, the Structured Interview for Prodromal Syndromes/Scale of Prodromal Symptoms (SIPS/SOPS) can be used to determine whether a candidate or patient has prodromal or symptomatic psychosis. To determine whether a candidate or patient has prodromal or symptomatic mania or hypomania, the Young Mania Rating Scale (YMRS), the Mania State Rating Scale (MSRS), the Bech-Rafaelsen Mania Scale (MAS), the Clinician-Administered Rating Scale for Mania (CARS-M), the Altman Self-Rating Mania Scale, or an equivalent variant thereof can be used, according to standard methods know in the art.

In some embodiments of any of the methods or systems described herein, the invention features one or more interventions or assessments. For example, an Ecological Momentary Intervention or Ecological Momentary Assessment (EMA) may be administered. EMAs include mood questionnaires, suicidality questionnaires, and psychopathology questionnaires. Mood questionnaires useful as part of the methods and systems of the invention include the Profile or Mood States (POMS), the Positive and Negative Affect Schedule (PANAS), and equivalent variants thereof. Suicidality questionnaires useful as part of the methods and systems of the invention include the Columbia Suicide Severity Rating Scale (C-SSRS) and equivalent variants thereof. Psychopathology questionnaires useful as part of the methods and systems of the invention include psychopathology questionnaires configured to assess hypomania or mania (e.g., the Hypomania/Mania Symptom Checklist (HCL-32), the Clinician-Administered Rating Scale for Mania (CARS-M), or the Young Mania Rating Scale (YMRS)) and psychopathology questionnaires configured to assess psychosis (e.g., the psychosis screening questionnaire, the Schizophrenia Test and Early Psychosis Indicator (STEPI), or the Cognitive Biases Questionnaire for psychosis (CBQp). Additionally or alternatively, any suitable EMA may be administered as part of the methods and systems of the invention provided herein.

An EMA may be administered upon certain conditions being met, or it may be administered automatically, e.g., at one or more predetermined time points, e.g., at regular intervals. In some instances, an EMA is administered in response to an increase in one or more behavioural characteristics indicative of onset of psychosis, mania, or hypomania. In some embodiments, an EMA is administered in response to an increase in a measure of risk of precipitating or exacerbating psychosis, mania, or hypomania.

The methods and systems of the invention provide a means to notify a third party (e.g., a clinician or pharmacy) if a patient undergoing treatment with a psychedelic therapy becomes non-compliant with the planned course of treatment. Systems of the invention can (i) receive and store information regarding the course of treatment with the psychedelic agent and (ii) receive and store inputs from a patient (e.g., actively acquired and passively acquired inputs) regarding dosing quantities and frequencies. By comparing planned dosing information to actual dosing information and characterizing the difference (e.g., based on a predetermined threshold), a system can detect patient compliance.

In some embodiments, the system passively acquires data (e.g., language data) that indicates that a psychedelic dose is too high (e.g., as a result of a non-compliant high administration or due to a greater-than-intended influence level of a planned dose). In any case, a third party may be notified to follow up with the patient, to temporarily suspend treatment, or both.

In embodiments in which the patient is being treated for a neurodegenerative disease, such as Alzheimer's disease, methods of the invention include conducting one or more cognitive assessments, which can detect drug-related brain decline. In some embodiments, cognitive assessments are conducted by methods known in the art, e.g., gold standard methods of assessments. In some embodiments, the cognitive assessment is a mini-mental state examination (MMSE), the Montreal cognitive assessment (MOCA), or the Alzheimer's disease assessment scale—cognitive subscale (ADAS-Cog).

Additionally or alternatively, methods of the invention may include obtaining a behavioral characteristic derived from an interaction between the patient and a device, e.g., using the methods and systems described in U.S. Pat. No. 9,474,481, which is incorporated herein by reference.

### System Architecture

The systems and methods of this invention can include or be implemented using any suitable processing system(s). Suitable processing systems include a computer based server (e.g., a remote server), a grid-computer system, a mainframe computer system, or a cloud-based computing platform. A processing system can be powered by any suitable computer processor (e.g. Intel or AMD processors), running operating systems such as Windows, Linux, Unix, or other standard operating systems. A server may have several gigabytes of random access memory. Servers can be connected to mass storage devices capable of storing gigabytes of data. The system and methods of the invention can be implemented in the form of software, such as database management software (e.g. SQL software which may run under popular database management programs such as MySQL or other systems such as Oracle), web server software such as Apache, programming languages for dynamic web pages such as PHP, Perl, Python, as well as other web application framework languages and systems languages such as Ruby on Rails, and the like. A server can be connected to suitable network, such as the internet, often by a high bandwidth connection.

In some embodiments, a network-connected computerized database contains a record of clinical support features, such as contact information for specialized treatment facilities, pharmacies, physicians, emergency personnel, and/or other support services. A system of the invention may include a software application (e.g., a patient-interface application, e.g., a mobile-device application) that accesses such a database conditionally or automatically to send one or more notifications, alerts, reports, or other information to a third party (e.g., to a computing platform (e.g., a remote database) or a clinical professional) for storage or analysis.

Various patient information can be stored on a processing system and/or shared with a third party, including data obtained from a screening session, such as raw data from a language sample (e.g., audio data, or text data), partially processed data obtained from a language sample (e.g., representation, summarization, or integration of a semantic analysis), a characteristic derived from processing a language sample (e.g., a semantic coherence measure, a syntactic complexity measure, a comprehension measure, a lexicon depth measure, or a lexicon breadth measure), a measure derived from one or more or such characteristics (e.g., a composite score derived from two or more of such characteristics (e.g., a composite of semantic coherence and syntactic complexity, or a measure of risk), or a relationship between any of the aforementioned data, e.g., obtained at different screening sessions, e.g., at different time points.

Behavioral data can also be stored on a processing system and/or shared with a third party. In some instances, such data includes social behavior data, mobility data, motion data, or any combination thereof.

Additionally or alternatively, personal identification information, medical records, and any messages or notes provided by the patient can be sent with a notification, alert, or report to a third party.

In some embodiments, the server database contains sufficient audio-video link information to establish audio and video communication between the candidate or patient and a clinician. For example, the server and database may contain both the internet address information for the practitioner, patient, and any third parties as needed, and also act to relay the data packets between the parties. Alternatively, the server and database may contain address links, such as, for example, for Skype or other online video conferencing systems enabling the patient, healthcare practitioner, and third parties to communicate by third party messaging systems. In general, in order to ensure quality and a consistent user interface, often the server will both present the telemedicine user interface (e.g. present one or more web pages for telemedicine applications) in addition to relaying the audio and video data packets. Accordingly, a telemedicine session can be suitably encrypted.

In any of the embodiments described above, the system can be configured to adhere to health-related privacy laws (e.g., HIPAA). For example, systems can be configured to privatize and/or anonymize individual data according to encryption protocols.

### Therapies

The methods of the invention can be used to assess the risk of developing psychosis, hypomania, or mania associated with a psychedelic therapy being administered for a variety of conditions. In some embodiments of the invention, the psychedelic therapy is being administered for treatment of a condition (e.g., a chronic condition), such as an inflammatory-related condition, a neurodegenerative condition, or a psychological condition. A psychedelic therapy can be administered as part of a complex therapy (i.e., a drug+non-drug therapy). For example, a psychedelic therapy can be administered as an adjunct therapy with a psychotherapy, such as talk therapy, existential therapy, or self-actualization therapy. The invention also includes psychedelic therapies and complex therapies occurring in a particular therapeutic setting, such as a specialized treatment facility, as described herein.

### Psychedelic Therapy

Using the methods and systems of the invention, a psychedelic agent can be administered on an in-patient or out-patient basis, or it can be self-administered under safe conditions using the methods and systems of the present invention. In some instances, a psychedelic agent is administered to a patient by a clinical professional. For example, the patient may be determined as likely to benefit from a perceptible dose (e.g., a medium to high dose) of the psychedelic therapy. In some embodiments, a perceptible dose of a psychedelic therapy is administered (e.g., a dose of greater than about 0.1 μg/kg, greater than about 0.5 μg/kg, greater than about 1.0 μg/kg, greater than about 5.0 μg/kg, greater than about 10 μg/kg, greater than about 20 μg/kg, greater than about 50 μg/kg, greater than about 100 μg/kg, greater than about 200 μg/kg, greater than about 500 μg/kg, greater than about 1.0 mg/kg, greater than about 5.0 mg/kg, greater than about 10 mg/kg, greater than about 50 mg/kg, or greater than about 100 mg/kg body weight, e.g., from about 0.1 μg/kg to about 0.5 μg/kg, from about 0.5 μg/kg to about 10 μg/kg, from about 1.0 μg/kg to about 5.0 μg/kg, from about 5.0 μg/kg to about 10 μg/kg, from about 10 μg/kg to about 50 μg/kg, from about 50 μg/kg to about 100 μg/kg, from about 100 μg/kg to about 500 μg/kg, from about 500 μg/kg to about 1.0 mg/kg, from about 1.0 mg/kg to about 10 mg/kg, from about 10 mg/kg to about 50 mg/kg, from about 50 mg/kg to about 100 mg/kg, or from about 100 mg/kg to about 500 mg/kg).

In some embodiments, a perceptible dose of psilocybin can be from 10 mg to 50 mg (e.g., from 10-25 mg, or from 25-50 mg, e.g., about 10 mg, about 15 mg, about 20 mg, about 25 mg, about 30 mg, about 35 mg, about 40 mg, about 45 mg, or about 50 mg). In some embodiments, a perceptible dose of LSD can be from 100 μg to 500 μg (e.g., from 100 μg to 200 μg, from 200 μg to 300 μg, from 300 μg to 400 μg, or from 400 μg to 500 μg, e.g., about 100 μg, about 150 μg, about 200 μg, about 250 μg, about 300 μg, about 350 μg, about 400 μg, about 450 μg, or about 500 μg).

In some instances, a perceptible dose of a psychedelic therapy is administered as an out-patient procedure, and the patient is monitored before release to ensure that any perceptible psychedelic effects (e.g., influences) have subsided. In this instance, the influence of the psychedelic therapy can be characterized at one or more (e.g., two, three, four, five, or more) time points following administration, e.g., to monitor its kinetics. For example, based on one or more of characteristics of a language sample obtained from a patient, a measure of influence can be derived using any of the methods described above. In some embodiments, a language sample is taken shortly after administration of the psychedelic agent (e.g., from 1-10 minutes, from 10-20 minutes, from 20-30 minutes, or within 1 hour, e.g., at 1 minute, 2 minutes, 3 minutes, 4 minutes, 5 minutes, 10 minutes, 15 minutes, 20 minutes, 25 minutes, 30 minutes, 40 minutes, or 50 minutes) to determine an influence measure at or near the psychedelic agent's peak effect. This peak influence measure may be compared to a reference measure (e.g., a baseline measure obtained from the same patient, or a measure derived from a plurality of subjects characterized as having a low influence measure or a low risk of developing psychosis, hypomania, or mania, e.g., as determined using any of the methods described herein). A subsequent language sample may be taken after any period of time from administration in which a psychedelic influence may have subsided (e.g., from 1-72 hours, e.g., from 24-72 hours or from 36-48 hours after administration, e.g., from 1-2 hours, from 2-3 hours, from 3-4 hours, from 4-5 hours, from 5-6 hours, from 6-7 hours, from 7-8 hours, from 8-10 hours, from 10-12 hours, from 12-14 hours, from 14-16 hours, form 16-18 hours, form 18-20 hours, from 20-22 hours, from 22-24 hours, from 24-36 hours, from 36-42 hours, from 42-48 hours, from 48-60 hours, or from 60-72 hours after administration, e.g., about 24 hours, about 36 hours, about 48 hours, about 60 hours, or about 72 hours after administration). The degree to which a psychedelic influence subsides can be characterized, for example, by a decrease from a peak influence measure to a subsequent influence measure.

In some instances, one or more language samples are elicited following administration of a psychedelic agent as part of an out-patient procedure to provide a final evaluation of the psychedelic effects on the patient. In some embodiments, the language sample is elicited by a structured interview with a clinical professional.

Alternatively, a psychedelic influence may be determined to have subsided by an alternative characterization method, for example, by comparing to a reference measure (e.g., a baseline measure obtained from the same patient, or measure derived from a plurality of subjects characterized as having a low influence measure or a low risk of developing psychosis, hypomania, or mania, e.g., as determined using any of the methods described herein). For example, an influence measure falling within a predetermined range (e.g., a range of error, e.g., a predetermined standard error) of a reference measure can used as a threshold below which a patient can be identified as having negligible or no residual psychedelic influence.

In other instances, the patient may be determined as likely to benefit from a sub-perceptible dose (e.g., a low dose) of the psychedelic therapy. In some embodiments of the invention, a sub-perceptible dose of a psychedelic therapy is administered (e.g., a dose of less than about 100 μg/kg, less than about 75 μg/kg, less than about 50 μg/kg, less than about 25 μg/kg, less than about 10 μg/kg, less than about 7.5 μg/kg, less than about 5.0 μg/kg, less than about 2.0 μg/kg, less than about 1.5 μg/kg, less than about 1.0 μg/kg, less than about 0.5 μg/kg, less than about 0.1 μg/kg, or less, e.g., from about 0.1 μg/kg to about 0.5 μg/kg, from about 0.5 μg/kg to about 10 μg/kg, from about 1.0 μg/kg to about 5.0 μg/kg, from about 5.0 μg/kg to about 10 μg/kg, from about 10 μg/kg to about 50 μg/kg, or from about 50 μg/kg to about 100 μg/kg). Sub-perceptible doses may, in some circumstances, be administered as an out-patient procedure.

Additionally or alternatively, a psychedelic therapy may self-administered by the patient (e.g., at a sub-perceptible dose). In some embodiments, the invention enables a patient to self-monitor their risk of developing psychosis, hypomania, or mania prior to or while undergoing a psychedelic therapy (e.g., a self-administered psychedelic therapy). In these instances, the systems of the invention can be equipped to relay results or implications of a patient's self-monitoring to a third party, such as a physician, pharmacist, administrative professional, a support professional, or another clinical professional.

In any of the above contexts, a course of administration may be altered according to a result of an assessment or characterization of a patient. In some embodiments, a psychedelic therapy is suspended if a patient is identified as at risk of developing psychosis, hypomania, or mania. Suspensions of therapy may be temporary (e.g., one or more days, weeks, months, or years, e.g., until a result is confirmed, until a patient is identified as having a low risk of developing psychosis, hypomania, or mania, or until a separate test independently qualifies the subject for treatment), or a suspension may be permanent. In some embodiments, a dose or frequency of a psychedelic therapy is decreased in response to identifying the patient as at risk of developing psychosis, hypomania, or mania. In some embodiments, the methods of the invention indicate that a patient's risk measure, or one or more characteristics of a language sample, suggest that the patient's risk of developing psychosis, hypomania, or mania is increasing, but have not reached a predetermined threshold. In such an instance, the dose or frequency of a psychedelic therapy may be decreased (e.g., temporarily decreased or permanently decrease). The invention allows for a dosage modification corresponding to the degree at which the patient's risk of developing psychosis changes.

### Patient Populations

The methods and systems of the present invention relate to assessment of patients who are undergoing therapy for a condition (e.g., alleviation of symptoms of a condition) or improvement of mental or physical well-being. Alternatively, the methods and systems of the invention relate to assessment of candidates for psychedelic therapy for a condition (e.g., alleviation of symptoms of a condition) or improvement of mental or physical well-being. A candidate for psychedelic therapy may be a patient undergoing a non-psychedelic therapy, such as a psychotherapy, who is being screened for suitability as a candidate for a complex therapy (i.e., a drug/non-drug therapy).

Thus, in some embodiments, the invention provides methods and systems for screening a patient who is undergoing psychedelic therapy for treatment of a condition (e.g., a chronic condition). In other instances, the invention provides methods and systems for screening a candidate for treatment of a condition (e.g., a chronic condition). The condition may be a chronic condition, e.g., a condition which may necessitate long-term psychedelic treatment, increasing the likelihood of developing psychosis, hypomania, or mania.

A psychedelic therapy (e.g., a 5-HT_{2A} receptor agonist (e.g., LSD, psilocybin, DOI, or (R)-DOI), an empathogenic agent (e.g., MDMA), or a dissociative agent (e.g., ketamine)) can be useful in treating a patient having an inflammatory condition, such as lung inflammation (e.g., chronic obstructive pulmonary disease (COPD)), neuroinflammation (e.g., Alzheimer's disease or dementia), rheumatoid arthritis, atherosclerosis, asthma (e.g., allergic asthma or non-allergic asthma), psoriasis, type II diabetes, inflammatory bowel disease, Crohn's disease, multiple sclerosis, and septicemia. For example, methods of treating Alzheimer's disease using a 5-HT_{2A} receptor agonist (e.g., LSD) are described, e.g., in International Patent Application No. WO 2016/145193, which is incorporated herein by reference. Methods of treating additional inflammatory conditions using psychedelic therapies (e.g., 5-HT_{2A} receptor agonists) are described, for example, in U.S. patent application Ser. No. 9,642,819, which is incorporated herein by reference.

In some embodiments, the patient is being administered a psychedelic agent for treatment of a condition associated with pathogenic ocular neovascularization, e.g., a human having macular degeneration (e.g., age-related macular degeneration), keratoconjunctivitis, conjunctivitis, keratitis, diabetic retinopathy, retinopathy of prematurity, polypoidal choroidal vasculopathy, ischemic proliferative retinopathy, retinitis pigmentosa, cone dystrophy, proliferative vitreoretinopathy, retinal artery occlusion, retinal vein occlusion, Leber's disease, retinal detachment, retinal pigment epithelial detachment, rubeosis iridis, corneal neovascularization, retinal neovascularization, choroidal neovascularization, retinochoroidal neovascularization, or a combination thereof.

Additionally or alternatively, patients and candidates that can be screened using the methods and systems of the invention include patients and candidates having a psychological condition treatable by a psychedelic therapy. Psychological conditions treatable by a psychedelic therapy (e.g., a 5-HT_{2A} receptor agonist (e.g., LSD, psilocybin, DOI, or (R)-DOI), an empathogenic agent, (e.g., MDMA), or a dissociative agent (e.g., ketamine)) include depression (e.g., major depression, melancholic depression, atypical depression, or dysthymia), an anxiety disorder (e.g., end of life anxiety, generalized anxiety disorder, panic disorder, social anxiety, post-traumatic stress disorder, acute stress disorder, obsessive compulsive disorder, or a social phobia), an addiction (e.g., a substance abuse or an eating disorder), a compulsive disorder (e.g., a primary impulse-control disorder or an obsessive-compulsive disorder), or a repetitive body-focused behavior (e.g., tic disorder or symptom thereof, such as Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder).

The methods and systems of the invention can be used to screen candidates and patients having a substance abuse problem, drug addiction, or other addictive behaviors. Addictive behaviors which can be treated using psychedelic therapy include food addiction, binge eating disorder, pathological gambling, pathological use of electronic devices, pathological use of electronic video games, pathological use of electronic communication devices, pathological use of cellular telephones, addiction to pornography, sex addiction, obsessive-compulsive disorder, compulsive spending, intermittent explosive disorder, kleptomania, pyromania, trichotillomania, compulsive over-exercising, and compulsive overworking. Drug addictions which can be treated using the methods of the invention include addictions to recreational drugs, as well as addictive medications. Examples of addictive agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine.

Additionally or alternatively, candidates and patients that can be screened using the methods and systems of the invention may have an anxiety disorder. Anxiety is broadly defined as a state of unwarranted or inappropriate worry often accompanied by restlessness, tension, distraction, irritability and sleep disturbances. This disproportionate response to environmental stimuli can hyperactivate the hypothalamic-pituitary-adrenal axis and the autonomic nervous system, resulting in somatic manifestation of anxiety, including shortness of breath, sweating, nausea, rapid heartbeat and elevated blood pressure. Anxiety disorders represent a range of conditions and as a result have been classified into multiple distinct conditions, including generalized anxiety disorder (GAD), panic disorder, social anxiety, post-traumatic stress disorder (PTSD), acute stress disorder (ASD), obsessive compulsive disorder (OCD), and social phobias.

GAD is the most commonly occurring of the anxiety disorders and is characterized by excessive and persistent worries. In the general population the lifetime prevalence rate of GAD ranges from 4.1 to 6.6% with somewhat higher rates in women than in men. The individual with GAD worries about life events such as marital relationships, job performance, health, money, and social status. Individuals with GAD startle easily and may suffer from depression. Some of the specific symptoms of GAD include restlessness, motor tension, difficulty concentrating, and irritability. The severity of the symptoms over time may be linked to the changing nature of the environmental stressor. With increasing age, GAD symptoms become less severe.

Panic disorder is a well-studied psychiatric condition that consists of multiple disabling panic attacks characterized by an intense autonomic arousal. In addition, heightened fear and anxiety states occur both during and between panic attacks. Approximately 3% of women and 1.5% of men have panic attacks. During a panic attack, the individual experiences multiple symptoms including light-headedness, a pounding heart and difficulty in breathing.

PTSD is a disorder characterized by intense fear and anxiety states that require psychiatric treatment. PTSD often results from exposure to a life threatening or traumatic event. Individuals with PTSD can have recurring thoughts of the terrifying event. Reenactment of the event varies in duration from a few seconds or hours to several days.

A psychedelic therapy can treat one or more symptoms (e.g., prodromal or somatic symptoms) of a psychological disorder. For example, a psychedelic therapy can treat a prodromal symptom of a depressive disorder (e.g., depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, or decreased initiation of voluntary responses). Additionally or alternatively, a psychedelic therapy can treat a somatic symptom, e.g., a somatic symptom associated with a psychological disorder (e.g., chronic pain, anxiety disproportionate to severity of physical complaints, pain disorder, body dysmorphia, conversion, hysteria, neurological conditions without identifiable cause, psychosomatic illness, or pain management in relation to an existing physical condition).

### Complex Therapy

In some embodiments, the methods of treatment and screening provided herein are performed in the context of an authorized treatment facility (e.g., a specialized treatment facility) configured to provide complex therapies to subjects in need thereof. Complex therapies may involve both pharmaceutical (e.g., psychedelic agent-based) and non-pharmaceutical treatments (e.g., behavioral therapy (e.g. cognitive behavioural therapy (CBT), brief behavioral activation for depression (BATD), talk therapy, existential therapy, and/or self-actualization therapy) designed according to a subject's specific needs. For example, methods provided herein enable a practitioner to determine whether a subject is likely to benefit from a psychedelic treatment and act accordingly. In many instances, a psychedelic treatment regimen may not be prescribed (and may be detrimental) outside the context of a specialized treatment facility in which a subject has access to adjunctive psychotherapy (e.g., behavioral therapy, existential, humanistic, or self-actualization therapy). Specialized treatment facilities can be configured to enhance the safety and efficacy of therapy (e.g., psychedelic therapy and/or complex therapy) through control and use of audio, visual, and other environmental factors. In general, specialized treatment facilities feature a staff that has training and expertise in administering and overseeing complex therapy, including psychedelic therapy and psychotherapy.

Treatment facilities in which psychedelic and/or complex therapies can be administered include other settings that are authorized to administer therapies including psychedelic therapies, adjunctive psychotherapies, and/or complex therapies. For example, authorized treatment facilities may be associated with a hospital, a mental health clinic, or a retreat center. Treatment facilities may be in-patient or out-patient facilities and may provide screening, evaluation, and follow-up services. In some embodiments, treatment facilities may be associated with a research facility/program.

A patient's response to therapy (e.g., psychedelic therapy, psychotherapy, and complex therapy) can be monitored and quantified using any suitable method known in the art according to the particular condition being treated.

In some cases, a complex therapy is provided to a patient via telemedicine, using systems described below.

### Psychedelic Agents

The invention features methods and systems involving a patient who is undergoing treatment with a psychedelic agent or who is a candidate for treatment with a psychedelic agent. In some embodiments, the invention involves monitoring patients undergoing treatment with psychedelic agents, e.g., for risk of precipitation or exacerbation of prodromal or symptomatic psychosis, mania, or hypomania.

The invention features methods related to treatment of psychedelic therapy. Psychedelic agents useful as part of the invention include any compound capable of inducing an altered state of consciousness, i.e., a marked deviation in the subjective experience or psychological functioning of a normal individual from his or her usual waking consciousness. Psychedelic agents include 5-HT_{2A} agonists (e.g., lysergic acid diethylamide (LSD), empathogenic agents (i.e., serotonin (5-HT) releasing agents; e.g., 3,4-methylenedioxymethamphetamine (MDMA)), and dissociative agents (i.e., N-Methyl-D-aspartate (NMDA) receptor agonists; e.g., ketamine).

5-HT_{2A} agonists include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl) methylamine; ayahuasca; 3,4,5-trimethoxyphenethylamine (mescaline); 5-methoxy-N,N-dimethyltryptamine (5-meo-DMT); and ibogaine.

In some embodiments, the 5-HT_{2A} agonist of the invention includes a compound having the following chemical formula (I):

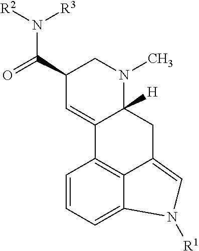

where exemplary values of the R groups in the above substituted chemical structure can be one or more of those represented in the following table:

| Name | R¹ | R² | R³ |
|---|---|---|---|
| LSD | H | CH₂CH₃ | CH₂CH₃ |
| Ergine | H | H | H |
| R-2-butyl | H | H | CH(CH₃)CH₂CH₃ |
| R-2-pentylamine | H | H | CH(CH₃)CH₂CH₂CH₃ |
| Analog of ergine | H | C₂H₅ | H |
| Analog of ergine | H | H | C₂H₅ |
| LSD | H | C₂H₅ | C₂H₅ |
| Analog of ergine | H | C₂H₅ | CH₂CH₂CH₃ |
| Analog of ergine | H | C₂H₅ | CH(CH₃)₂ |
| Analog of ergine | H | CH₂CH₂CH₃ | H |
| Analog of ergine | H | H | CH₂CH₂CH₃ |
| Analog of ergine | H | CH₂CH₂CH₃ | CH₂CH₂CH₃ |
| Analog of ergine | H | CH₂CH₂CH₃ | C₂H₅ |
| Analog of ergine | H | CH₂CH₂CH₃ | CH(CH₃)₂ |
| Analog of ergine | H | CH(CH₃)₂ | H |
| Analog of ergine | H | H | CH(CH₃)₂ |
| Analog of ergine | H | CH(CH₃)₂ | CH(CH₃)₂ |
| Analog of ergine | H | CH(CH₃)₂ | C₂H₅ |
| Analog of ergine | H | CH(CH₃)₂ | CH₂CH₂CH₃ |

In some embodiments, R¹ of formula (I) can be H, C₁-C₆-alkyl, OH, O—(C₁-C₆-alkyl), halogen, or C₁-C₄-haloalkyl; R² of formula (I) can be H, C₁-C₆-alkyl, OH, O—(C₁-C₆-alkyl), halogen, or C₁-C₄-haloalkyl; and R³ of formula (I) can be H, C₁-C₆-alkyl, OH, O—(C₁-C₆-alkyl), halogen, or C₁-C₄-haloalkyl.

In some embodiments, the 5-HT_{2A} agonist of the invention includes a compound having the following chemical formula (II):

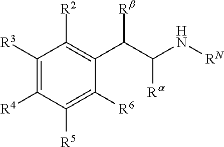

where exemplary values of the R groups in the above substituted chemical structure can be one or more of those represented in the following table:

| Name | R² | R³ | R⁴ | R⁵ | R^{α} | R^{β} |
|---|---|---|---|---|---|---|
| Mescaline | | OCH₃ | OCH₃ | OCH₃ | | |
| TMA | | OCH₃ | OCH₃ | OCH₃ | CH₃ | |
| TMA-2 | OCH₃ | | OCH₃ | OCH₃ | CH₃ | |
| methoxyDOB | OCH₃ | | Br | OCH₃ | CH₃ | OCH₃ |
| DOM | OCH₃ | | CH₃ | OCH₃ | | |
| DOB | OCH₃ | | Br | OCH₃ | | |
| DOI | OCH₃ | | I | OCH₃ | | |
| Sulfur analog of | | OCH₃ | OCH₃ | SCH₃ | | |
| mescaline | | | | | | |
| Sulfur analog of | | OCH₃ | SCH₃ | OCH₃ | | |
| mescaline | | | | | | |
| DOIB | OCH₃ | | CH₂CH(CH₃)₂ | OCH₃ | CH₃ | |
| DOTFM | OCH₃ | | CF₃ | OCH₃ | CH₃ | |

In some embodiments, R² of formula (II) can be OH, O—(C₁-C₆-alkyl), —O—(C₂-C₆-alkyl)-N(R⁵)₂, or —O—(C₂-C₆-alkyl)-N(R^{x})₃+halogen⁻; R³ of formula (II) can be OH, O—(C₁-C₆-alkyl), —O—(C₂-C₆-alkyl)-N(R^{x})₂, or —O—(C₂-C₆-alkyl)-N(R^{x})₃⁺halogen⁻; R⁴ of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃+halogen⁻; R⁵ of formula (II) can be halogen, C₁-C₂-haloalkyl, H, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃+halogen⁻; R⁶ of formula (II) can be halogen, C₁-C₂-haloalkyl, H, C₁-C₆-alkyl, —S—(C₁-C₆-alkyl), OH, O—(C₁-C₆-alkyl), —O—(C₂-C₆-alkyl)-N(R⁵)₂, or —O—(C₂-C₆-alkyl)-N(R⁵)₃⁺halogen⁻; R^{α} is H, halogen, or C₁-C₆-alkyl; R^{β} of formula (II) can be OH, O—(C₁-C₆-alkyl), —O—(C₂-C₆-alkyl)-N(R⁵)₂, or —O—(C₂-C₆-alkyl)-N(R^{x})₃+halogen⁻; R^{N} of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{x})₂, or —O—(C₂-C₆-alkyl)-N(R^{x})₃⁺halogen⁻; and R^{x} is independently H or C₁-C₄-alkyl.

In some embodiments, the 5-HT_{2A} agonist of the invention includes a compound having the following chemical formula (III):

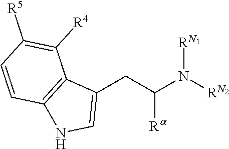

where exemplary values of the R groups in the above substituted chemical structure can be one or more of those represented in the following table:

| Name | R^{N}₁ | R^{N}₂ | R^{α} | R⁴ | R⁵ | R⁶ | R⁷ |
|---|---|---|---|---|---|---|---|
| 6-fluoro-psilocin | C | C | H | OH | H | F | H |
| 7-fluoro-psilocin | C | C | H | OH | H | H | F |
| 4-fluoro-5-methoxy- | C | C | H | F | OCH₃ | H | H |
| DMT | | | | | | | |
| 6-fluoro-5-methoxy- | C | C | H | H | OCH₃ | F | H |
| DMT | | | | | | | |
| α-Methyl-tryptamine | H | H | CH₃ | H | H | H | H |
| Serotonin | H | H | H | H | OH | H | H |

In some embodiments, R^{N}₁ of formula (III) can be H, C₁-C₆-alkyl, OH, O—(C₁-C₆-alkyl), halogen, or C₁-C₄-haloalkyl; R^{N}₂ of formula (III) can be H, C₁-C₆-alkyl, OH, O—(C₁-C₆-alkyl), halogen, or C₁-C₄-haloalkyl; R^{α} of formula (III) can be H, C₁-C₆-alkyl, OH, O—(C₁-C₆-alkyl), halogen, or C₁-C₄-haloalkyl; R⁴ of formula (III) can be H, C₁-C₆-alkyl, OH, O—(C₁-C₆-alkyl), halogen, or C₁-C₄-haloalkyl; R⁵ of formula (III) can be H, C₁-C₆-alkyl, OH, O—(C₁-C₆-alkyl), halogen, or C₁-C₄-haloalkyl; R⁶ of formula (III) can be H, C₁-C₆-alkyl, OH, O—(C₁-C₆-alkyl), halogen, or C₁-C₄-haloalkyl; and R⁷ of formula (III) can be H, C₁-C₆-alkyl, OH, O—(C₁-C₆-alkyl), halogen, or C₁-C₄-haloalkyl.

### Formulations

Formulations of psychedelic agents for oral use include tablets containing the psychedelic agent in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. For example, the coating may be adapted to release a psychedelic agent in a predetermined pattern (e.g., in order to achieve a controlled release formulation) or it may be adapted not to release the psychedelic agent until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the psychedelic agent). The coating may be applied on the solid dosage form in a similar manner as that described in Encyclopedia of Pharmaceutical Technology (eds. J. Swarbrick and J. C. Boylan, 1988-1999, Marcel Dekker, New York).

Formulations for oral use may also be presented as chewable tablets, or as hard gelatin capsules wherein the psychedelic agent is mixed with an inert solid diluent (e.g., potato starch, lactose, microcrystalline cellulose, calcium carbonate, calcium phosphate or kaolin), or as soft gelatin capsules wherein the psychedelic compound is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Powders, dispersible powders, or granules suitable for preparation of an aqueous suspension by addition of water are convenient dosage forms for oral administration of psychedelic agents. Formulation as a suspension provides the psychedelic agent in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Hayes (Remington: The Science and Practice of Pharmacy, volume I and volume II. Twenty-second edition. Philadelphia, 2012).

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the psychedelic compound, the composition may include suitable parenterally acceptable carriers and/or excipients. The psychedelic agent may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the psychedelic agent is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Examples

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1. Screening a Psilocybin Therapy Patient for Risk of Developing Psychosis

A 35-year old patient diagnosed with major depression has scored 17 on the 29-item Hamilton Rating Scale for Depression (HRSD) and is identified as a candidate for a complex therapy featuring in-patient administration of psilocybin in combination with adjunctive behavior therapy. Utilizing a mobile device operated by a clinician responsible for screening patients for eligibility to receive the complex therapy, the patient is instructed to describe a recent dream they have had, or to react to an image or photograph known to elicit a negative emotional response, and this response is recorded, pre-processed, and transmitted via the mobile device to a remote server for analysis. Alternatively, this assessment is conducted remotely by a screening clinician via computer or mobile device, or via an automated screening application delivered via computer or mobile device. The patient is notified by the screening clinician during the visit or in the hours or days following the screening visit if they are eligible to receive the complex therapy based upon an assessment of their speech provided by the remote server. Alternatively, the patient is notified via email, text message, or other means of electronic notification of their eligibility to receive the complex therapy.

Should the results of the automated analysis indicate that the patient is at low risk to developing psychosis, hypomania, or mania, the patient would be invited to receive the complex therapy, and likewise, should such analysis indicate that the patient is at high risk to developing psychosis, hypomania, or mania, the patient would not be invited to receive the complex therapy. Regardless of the outcome of the automated analysis, the results would be reported to the primary care physician or appropriate clinician overseeing psychiatric care for the patient.

### Example 2. On-Going Safety Monitoring of LSD Therapy Patient for Risk of Developing Psychosis

After agreeing to adhere to a smartphone-based screening program as a follow-on component of the complex therapy involving LSD and behavior therapy, a 54-year old patient diagnosed with substance abuse is administered LSD at a treatment center. The patient downloads a software application to her smartphone at the treatment center, is instructed on the proper and regular use of the application, and is notified via push notifications when her mobile device safety session is scheduled, e.g., monthly, beginning one day after the complex therapy is administered.

Utilizing the smartphone-based safety monitoring application, the patient is digitally prompted to speak freely about a recent experience and her expectations for the future. Several follow-on prompts are provided to generate a twenty-minute audio recording, which is pre-processed on the smartphone processor and sent to a remote server for language analysis. Using methods such as latent semantic analysis, and by comparing the resulting measure of risk to her own baseline speech previous to treatment administration, the remote server determines that the subject has a low measure of risk for developing psychosis, hypomania, or mania. The server sends a communication to the patient's smartphone enabling the software application to continue to prompt the patient to provide language samples at the predetermined interval. The patient undergoes adjunctive behavioral therapy through the same smartphone application in parallel with the safety assessment.

One week after being administered the complex therapy, the patient speech samples are determined to indicate that the patient is at high risk for developing psychosis, based on automated assessments of multiple speech samples taken at varying time points (1 day, 3 days, 7 days) following the complex therapy. The remote server tasked with assessing speech samples sends a communication to the patient's smartphone notifying her that a clinician will be in contact to follow up. A second communication is sent to a clinician, including a summary report of the results of the assessments and the statistical confidence associated with the risk assessment. The clinician would direct the patient to either provide an additional speech sample for further validation, or direct the client to an appropriate mental health clinic for further assessment and or therapy based upon the summary report of safety assessment.

### Example 3. In-Patient Administration of R-DOI for Acute Inflammatory Condition

A 65-year old patient is diagnosed with herpetic encephalitis, is hospitalized due to the life-threatening nature of the condition, and requires immediate treatment with anti-inflammatory therapy. Following administration of R-DOI, the patient's encephalitis is successfully treated and he is evaluated by doctors for release from the hospital. Utilizing a mobile device operated by clinicians at the hospital, clinicians instruct the patient to describe a recent dream he has had, or to react to an image or photograph known to elicit a negative emotional response, and capture his response via the mobile device for pre-processing and transmission to a remote server for analysis. Using methods described herein, the remote server determines that the patient is at low risk for developing psychosis, hypomania, or mania and is released from the hospital once this result is transmitted back to supervising clinicians at the hospital. Should the patient have a moderate to high measure or risk for developing psychosis, hypomania, or mania, as determined through statistical confidence interval, the patient would either be kept at the hospital for further observation by clinicians, or would agree to adhere to a smartphone-based screening program that would allow clinicians to remotely assess the patient's risk for developing psychosis, hypomania, or mania.

### Example 4. Monitoring LSD Therapy in Alzheimer's Disease (AD) Patients

AD patients are susceptible to psychosis, particularly in late stages of associated neurodegeneration. As previously described, LSD may be administered at very low doses (e.g., sub-perceptible doses) as a means of alleviating symptoms of AD and modifying the disease progression, but may also pose a risk to patients that initiate this therapy at a stage of the disease in which the risk for psychosis is heightened. Utilizing the technology described herein, a clinician remotely monitors patients for side-effects associated with chronic low dose administration of LSD to enhance patient safety. Should a speech or text sample provided by an AD patient via smartphone or other mobile device be assessed via automated processing and analysis indicate that the patient is either at high risk to or currently experiencing psychosis, the patient's primary care provider (or emergency contact) and the patient's primary care physician would be notified. Based upon this notification, the patient's primary care clinician would take appropriate and immediate additional steps to either evaluate the patient's condition, and/or modify or discontinue the patient's use of low dose LSD for the alleviation of symptoms associated with AD and/or its disease progression, and/or treat the patient with appropriate psychiatric medication (i.e. Olanzapine or other anti-psychotic) to treat the symptoms of psychosis, or prevent psychosis from manifesting.

### Example 5. Monitoring the Safety of Drug Therapy in Asthma Patients

Asthma patients typically are advised to self-administer control medications for the prevention of asthma exacerbation on a periodic basis. As previously described, R-DOI may be administered to patients for the control of asthma. Psychiatric side-effects may manifest in asthma patients administered R-DOI or other serotonin 5-HT_{2A} receptor agonist, including mania and psychosis. Utilizing the technology described herein, a clinician remotely monitors patients for side-effects associated with the use of R-DOI for the control of asthma. Should a speech or text sample provided by a patient utilizing R-DOI for the control of asthma be assessed via automated processing and analysis indicate that the patient is either at risk to or currently experiencing psychosis, the patient's primary care provider (or emergency contact) and the patient's primary care physician would be notified. Further, the patient's primary care clinician and could thereby take appropriate immediate additional steps to either evaluate the patient's condition, and/or modify or discontinue the patient's use of R-DOI for the control of asthma and/or its disease progression, and treat the patient with appropriate psychiatric medication to address symptoms associated with psychosis, hypomania, or mania.

### Example 6. Administration of Ketamine in an Out-Patient Facility

Ketamine and esketamine are used as anti-depressant therapies for use in severe major depression. Use of the invention in the context of using ketamine or esketamine for the treatment of depression involves application as both a screening tool, to exclude patients at risk for psychosis, hypomania, or mania, as well as in the release interview for the patient, enabling clinicians to confirm the drug effects have subsided and the absence of lingering psychotic or manic symptoms.

### Example 7. Screening Prior to Chronic Treatment with Low-Dose LSD

A 70-year old patient with mild cognitive impairment is a candidate for chronic LSD treatment for alleviating symptoms of cognitive decline. At screening, the patient is asked to describe a recent dream, or to react to an image known to elicit a negative emotional response. His response is captured via the mobile device and analyzed based on population-wide features indicative of psychosis, hypomania, or mania. Using the methods described herein, the software outputs the level of risk of this patient of developing psychosis, hypomania, or mania. The clinician responsible for screening who is sent the report immediately, would further evaluate the patient's condition and determine the suitability of the subject for treatment based on level of risk.

### Example 8. Patient-Specific Risk Assessment

A 60-year old individual with mild cognitive impairment is a candidate for chronic LSD treatment. At screening via a smartphone application, the patient is digitally prompted to react to an image or photograph known to elicit a negative emotional response. His response is captured via the mobile device and is analyzed locally on the device by comparing to known properties of normal and abnormal acoustic variance and/or semantic coherence. The system determines that the candidate is not likely to develop psychosis, hypomania, or mania, and a report qualifying the candidate for the LSD treatment is sent to the pharmacy (e.g., directly or indirectly, e.g., by uploading the report to a database accessible to a pharmacy).

The patient continues to provide speech samples to the smartphone application on a daily basis. The speech samples are analyzed by a local processor within the smartphone device (i.e., without sending to a remote server). Characteristics derived from the speech samples are analyzed and compared to the patient's prior samples, including the baseline characteristics derived from the qualifying sample provided at screening. Thus, the system utilizes a patient-specific risk assessment methodology.

On the third week of using the program, the patient fails to respond to the speech sample acquisition prompt for three consecutive days. As a result, the application is triggered to generate a cumulative report of the speech sample data since beginning the program and sends the report to the subject's clinician with instructions to follow-up with the patient.

### Example 9. Computer Systems

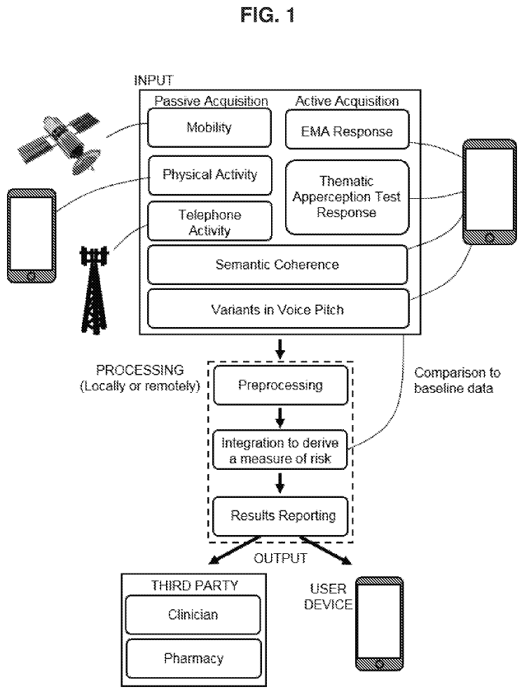

The invention provides computer systems for executing any of the methods provided herein. One such system is shown in FIG. 1. The top box contains several exemplary inputs, which can be acquired using passive or active acquisition mechanisms. Passive acquisition involves constant or arbitrary measurements of a subject's whereabouts, physical activity, and telephone activity, for example. Mobility (i.e., change in whereabouts) is monitored through GPS. Physical activity is monitored using an accelerometer integrated within a smartphone. Telephone activity is obtained by a telecommunications provider. Active acquisition is input that is elicited, for example, by a clinician or by a software program. Active acquisition includes responses to questionnaires, such as an EMA, or responses to a thematic apperception test, both of which are administered, in this example, automatically, as part of a software program on the user's smartphone device. Language samples, including characteristics such as semantic coherence and variants in a user's voice pitch, can be acquired either passively or actively, or both, using the smartphone's microphone.

As shown in the middle, dashed box, once the language samples and/or behavioral samples are recorded, they can be processed (e.g., converted from raw data into an individual language or behavioral characteristic), integrated with one or more other characteristics obtained from the subject to derive a measure of risk, and reported. This step can be performed locally (e.g., on the smartphone itself) or remotely (e.g., after being sent as an encrypted file to a remote server or by cloud computing). When performed locally, the integration and derivation step includes a comparison to the subject's baseline data, obtained at an earlier time point. When performed remotely, the integration and derivation step may involve comparison with a pool of data derived from individuals, e.g., in real time. Alternatively, the system can be configured to perform the processing step locally at some times and remotely at others (e.g., at predetermined intervals or contingent upon wireless network availability).

As discussed above, the computer system includes a mechanism to send a report to a third party and/or to view all or a portion of the results on the application's interface to the subject.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of screening a candidate for treatment with a psychedelic agent, the method comprising:
(i) obtaining a language sample from a treatment candidate, wherein the candidate has not begun treatment with a psychedelic agent;
(ii) deriving one or more language characteristics from the language sample; and
(iii) based on the one or more language characteristics, determining a measure of risk, wherein the measure of risk correlates with a risk of precipitating or exacerbating psychosis, hypomania, or mania in the candidate.

2. The method of claim 1, further comprising sending a report to a third party.

3. The method of claim 2, wherein the third party is a clinical professional.

4. The method of claim 3, wherein the clinical professional is a physician, pharmacist, administrative professional, nurse, support professional, or caretaker.

5. The method of claim 2, wherein the third party is a computing platform.

6. The method of any one of claims 2-5, wherein the report informs a decision to prescribe or administer the psychedelic therapy.

7. The method of any one of claims 2-6, wherein the report informs a dosing regimen for the psychedelic therapy.

8. The method of any one of claims 1-7, wherein the candidate has been characterized as unlikely to have or develop paranoid ideation, paranoid personality disorder, a personality disorder, an intellectual disability, or bipolar disorder.

9. The method of any one of claims 1-7, further comprising screening the candidate for a likelihood of having or developing paranoid ideation, paranoid personality disorder, a personality disorder, an intellectual disability, or bipolar disorder.

10. A method of reducing a risk of developing psychosis, hypomania, or mania in a patient undergoing treatment with a psychedelic agent, the method comprising:
(i) obtaining a language sample from the patient undergoing treatment with a psychedelic agent;
(ii) deriving one or more characteristics of the language sample;
(iii) based on the one or more characteristics, determining a measure of risk, wherein the measure of risk correlates with the risk of precipitating or exacerbating psychosis, hypomania, or mania in the patient; and
(iv) based on the measure of risk, recommending whether to suspend the treatment with the psychedelic agent.

11. The method of claim 10, further comprising sending a report to a third party.

12. The method of claim 11, wherein the third party is a clinical professional.

13. The method of claim 12, wherein the clinical professional is a physician, pharmacist, administrative professional, nurse, support professional, or caretaker.

14. The method of claim 11, wherein the third party is a computing platform.

15. A method of assessing a risk of precipitating or exacerbating psychosis, hypomania, or mania in a patient undergoing treatment with a psychedelic agent, the method comprising:
(i) providing a plurality of measures of risk, each measure of risk derived from one or more characteristics of a language sample obtained from the patient, wherein each measure of risk is associated with a different treatment time point; and
(ii) comparing two or more of the plurality of measure of risk to obtain a differential measure of risk, wherein the patient is identified as at risk of precipitating or exacerbating psychosis, hypomania, or mania if the differential measure of risk exceeds a predetermined threshold.

16. The method of claim 15, further comprising sending one or more reports to a third party.

17. The method of claim 16, wherein the third party is a clinical professional.

18. The method of claim 17, wherein the clinical professional is a physician, pharmacist, administrative professional, nurse, support professional, or caretaker.

19. The method of claim 18, wherein the third party is a computing platform.

20. The method of claim 19, wherein the one or more reports recommends suspending the treatment with the psychedelic agent if the differential risk measure exceeds the predetermined threshold.

21. A method of providing a regimen of psychedelic therapy to a patient, the method comprising:
(i) providing a differential measure of risk obtained by comparing two or more measures of risk, each measure of risk derived from one or more language characteristics of a language sample obtained from the patient, wherein the one or more measures of risk correlates with the risk of precipitating or exacerbating psychosis, hypomania, or mania in the patient, and wherein each measure of risk is associated with a different treatment time point; and
(ii) suspending the psychedelic therapy if the differential measure of risk exceeds a predetermined threshold.

22. The method of any one of claims 10-21, wherein the patient has been screened for one or more adverse effects associated with the psychedelic agent, wherein the one or more adverse effects are selected from the group consisting of depersonalization, dissociation, derealization, hallucinogenic or psychoactive abuse, a hallucinogen-use disorder, a hallucinogen-induced disorder, a high-risk behavior, and mania.

23. The method of any one of claims 10-21, further comprising screening the patient for one or more adverse effects associate with the psychedelic agent, wherein the one or more adverse effects are selected from the group consisting of depersonalization, dissociation, derealization, hallucinogenic or psychoactive abuse, a hallucinogen-use disorder, a hallucinogen-induced disorder, a high-risk behavior, and mania.

24. The method of claim 22 or 23, further comprising administering the psychedelic agent if the screening indicates that the patient is not experiencing the one or more adverse effects.

25. The method of any one of claims 10-24, further comprising assessing a measure of compliance with, or abuse of, the psychedelic agent.

26. The method of claim 25, wherein the measure of compliance with, or abuse of, the psychedelic agent is derived from a biomarker.

27. The method of any one of claims 10-26, further comprising determining a frequency of retreatment of the patient with the psychedelic agent, wherein the frequency of retreatment is determined by:
(i) providing a measure of efficacy correlated with a positive therapeutic response in the patient to the psychedelic agent;
(ii) providing a measure of risk correlated with a risk of precipitating or exacerbating a disease state associated with stress or a psychopathology; and
(iii) based on steps (i) and (ii), determining a frequency of retreatment with the psychedelic agent, wherein the measure of efficacy and/or the measure of risk is an output from a clinical assessment.

28. A method of determining a frequency of retreatment of a patient with a psychedelic agent, the method comprising:
(i) providing a measure of efficacy correlated with a positive therapeutic response in the patient to the psychedelic agent;
(ii) providing a measure of risk correlated with a risk of precipitating or exacerbating a disease state associated with stress or a psychopathology; and
(iii) based on the measure of efficacy and the measure of risk, determining a frequency of retreatment with the psychedelic agent, wherein the measure of efficacy and/or the measure of risk is an output from a clinical assessment.

29. A method of retreating or redosing a patient for a disease or disorder for which the patient is being treated or has been previously treated, the method comprising:
(i) detecting an increase in one or more symptoms of a condition in the patient, wherein the patient has undergone a digital clinical assessment to obtain a language characteristic, a behavioral characteristic, and/or a biomarker; and
(ii) retreating or redosing the patient for the condition.

30. The method of claim 29, wherein the condition is associated with deterioration of mental health.

31. The method of claim 27 or 28, wherein one or more factors of the clinical assessment comprise a language characteristic, a behavioral characteristic, and/or a biomarker.

32. The method of claim 30 or 31, wherein the frequency of retreatment is from bi-weekly to annually.

33. The method of any one of claims 10-26, further comprising adjusting the dose and/or frequency of treatment with the psychedelic agent based on one or more behavioral characteristics, language characteristics, and/or biomarkers.

34. A method of administering a psychedelic agent to a patient in need thereof, the method comprising:
(i) obtaining one or more measures of risk derived from one or more language characteristics of a language sample obtained from the patient, wherein the one or more measures of risk correlates with the risk of precipitating or exacerbating psychosis, hypomania, or mania in the patient; and
(ii) administering the psychedelic agent if the measure of risk is below a predetermined threshold.

35. A method of characterizing the influence of a psychedelic agent on the perception of a patient administered therewith, the method comprising:
(i) obtaining a language sample from the patient;
(ii) providing one or more language characteristics of the language sample; and
(ii) based on the one or more language characteristics, determining a measure of psychedelic influence, wherein the measure of psychedelic influence correlates with the influence of the psychedelic compound on the perception of the patient.

36. The method of any one of claims 1-35, further comprising, in response to determining that a candidate has a high measure of risk, prompting an ecological momentary assessment (EMA) of the candidate.

37. The method of any one of claims 1-36, wherein the language sample is elicited by a digital prompt, a questionnaire, or a clinician administered interview.

38. The method of any one of claims 1-37, wherein the language sample is a dream report, a description of a picture, a thematic apperception test, or a neutral text reading.

39. The method of any one of claims 1-38, wherein the language sample is obtained by passive acquisition.

40. The method of any one of claims 1-39, wherein the language sample is a text sample.

41. The method of any one of claims 1-40, wherein the language sample is an audio sample.

42. The method of claim 41, wherein the audio sample is analyzed to derive the one or more language characteristics, wherein the one or more language characteristics comprises one or more acoustic features.

43. The method of claim 42, wherein the one or more acoustic features are selected from the group consisting of a measure of irregular pitch, zero-crossing rate, kurtosis energy, harmonics-to-noise ratio (HNR), mel-frequency cepstral coefficients (MFCC), and frame energy.

44. The method of any one of claims 41-43, wherein the audio sample is transcribed into text.

45. The method of any one of claims 1-44, wherein the language sample is analyzed to derive the one or more language characteristics, wherein the one or more language characteristics comprises a measure of semantic coherence.

46. The method of claim 45, wherein a low measure of semantic coherence is positively correlated with the risk of developing psychosis.

47. The method of any one of claims 1-46, wherein the language sample is analyzed to derive the one or more language characteristics, wherein the one or more language characteristics comprises a measure of syntactic complexity.

48. The method of claim 41, wherein a low measure of syntactic complexity is positively correlated with the risk of developing psychosis.

49. The method of any one of claims 1-48, wherein the language sample is analyzed to derive the one or more language characteristics, wherein the one or more language characteristics comprises a measure of maximum phrase length.

50. The method of claim 49, wherein a low measure of maximum phrase length is positively correlated with the risk of developing psychosis.

51. The method of any one of claims 1-50, wherein the language sample is analyzed to derive the one or more language characteristics, wherein the one or more language characteristics comprises a measure of lexicon breadth or depth.

52. The method of claim 51, wherein a high measure of lexicon breadth or depth is positively correlated with the risk of developing hypomania or mania and/or a low measure of lexicon breadth or depth is positively correlated with the risk of developing psychosis.

53. The method of any one of claims 1-52, wherein the language sample is analyzed to derive the one or more language characteristics, wherein the one or more language characteristics comprises a measure of logorrhea.

54. The method of claim 53, wherein a high measure of logorrhea is positively correlated with the risk of developing hypomania or mania.

55. The method of any one of claims 1-54, wherein the language sample is analyzed to derive the one or more language characteristics, wherein the one or more language characteristics comprises a measure of psychometrics.

56. The method of claim 55, wherein the measure of psychometrics is latent inhibition.

57. The method of claim 56, wherein a low measure of latent inhibition is positively correlated with the risk of developing psychosis, hypomania, or mania.

58. The method of any one of claims 1-57, wherein the language sample is analyzed to derive speech graph attributes.

59. The method of claim 58, wherein the speech graph attributes are input to a machine learning algorithm.

60. The method of any one of claims 1-59, wherein the language sample is analyzed to derive the one or more language characteristics, wherein the one or more language characteristics comprises a measure of flight of thought.

61. The method of claim 60, wherein a high measure of flight of thought is positively correlated with the risk of developing hypomania or mania.

62. The method of any one of claims 1-61, wherein the language sample is analyzed to derive the one or more language characteristics, wherein the one or more language characteristics comprises a measure of recursiveness.

63. The method of claim 62, wherein a high measure of recursiveness is positively correlated with the risk of developing hypomania or mania.

64. The method of any one of claims 1-63, wherein the measure of risk is further based on one or more behavioral characteristics.

65. The method of claim 64, wherein the one or more behavioral characteristics are derived from a telephone record.

66. The method of claim 65, wherein the one or more behavioral characteristics derived from a telephone record comprise a number or frequency of outgoing calls or messages, a number or frequency of incoming calls or messages, a ratio between a number or frequency of outgoing calls or messages and a number or frequency of incoming calls or messages, a duration of one or more calls, a length of one or more messages, a number or frequency of newly added contacts, a number of changes in cell tower IDs, or a number of unique cell tower IDs.

67. The method of claim 66, wherein a number or frequency of outgoing calls or messages is positively correlated with the risk of developing hypomania or mania.

68. The method of claim 66 or 67, wherein a ratio between a number or frequency of outgoing calls or messages and a number or frequency of incoming calls or messages is positively correlated with the risk of developing hypomania or mania.

69. The method of any one of claims 66-68, wherein a duration of one or more calls is positively correlated with the risk of developing hypomania or mania.

70. The method of any one of claims 66-69, wherein the length of one or more messages is positively correlated with the risk of developing hypomania or mania.

71. The method of any one of claims 66-70, wherein a number of unique phone numbers is positively correlated with the risk of developing hypomania or mania.

72. The method of any one of claims 1-71, wherein the one or more behavioral characteristics comprise a number or frequency of instances in which a mobile device screen is turned on.

73. The method of any one of claims 1-72, wherein the one or more behavioral characteristics comprise a measure of activity detected by a sensor.

74. The method of claim 73, wherein the sensor is an antenna on a mobile device.

75. The method of claim 73 or 74, wherein the sensor is in communication with a global positioning system (GPS).

76. The method of any one of claims 73-75, wherein the measure of activity is a measure of mobility.

77. The method of claim 76, wherein a high measure of mobility is positively correlated with the risk of developing hypomania or mania.

78. The method of claim 73, wherein the sensor is an accelerometer.

79. The method of claim 78, wherein the measure of activity comprises a measure of movement.

80. The method of claim 79, wherein a measure of movement is positively correlated with the risk of developing hypomania or mania.

81. The method of claim 64, wherein the one or more behavioral characteristics are derived from a frequency, duration, or quality of sleep.

82. The measure of claim 81, wherein the measure of frequency, duration, or quality of sleep is derived from a frequency and/or duration of light exposure.

83. The method of claim 64, wherein the one or more behavioral characteristics are derived from:
(a) speed of typing: and/or
(b) one or more human-computer interactions selected from the group consisting of swipes, taps, and keystroke events.

84. The method of any one of claims 73-83, wherein the sensor is or is in communication with a wireless network hub.

85. The method of any one of claims 36-84, wherein the measure of risk is further based on a result of the EMA.

86. The method of claim 85, wherein the measure of risk refers to a risk or precipitating or exacerbating hypomania or mania, and wherein the EMA is a psychopathology questionnaire configured to assess hypomania or mania.

87. The method of claim 86, wherein the EMA is the Hypomania/Mania Symptom Checklist (HCL-32), the Clinician-Administered Rating Scale for Mania (CARS-M), the Young Mania Rating Scale (YMRS), or an equivalent variant thereof.

88. The method of claim 85, wherein the measure of risk refers to a risk of precipitating or exacerbating psychosis, and wherein the EMA is a psychopathology questionnaire configured to assess psychosis.

89. The method of claim 88, wherein the EMA is the psychosis screening questionnaire, the Schizophrenia Test and Early Psychosis Indicator (STEPI), the Cognitive Biases Questionnaire for psychosis (CBQp), or an equivalent variant thereof.

90. A method of characterizing the influence of a psychedelic agent on the perception of a patient administered therewith, the method comprising:
(i) obtaining a language sample from the patient;
(ii) providing one or more language characteristics of the language sample; and
(ii) based on the one or more language characteristics, determining a measure of psychedelic influence, wherein the measure of psychedelic influence correlates with the influence of the psychedelic therapy on the perception of the patient.

91. The method of claim 90, further comprising providing a notification based on the influence of a psychedelic agent on the perception of the patient.

92. The method of claim 90 or 91, wherein the psychedelic agent is administered on an in-patient basis.

93. The method of claim 92, wherein the psychedelic agent is administered in a perceptible dose.

94. The method of claim 92 or 93, wherein the psychedelic agent is administered on an out-patient basis.

95. The method of claim 94, wherein the psychedelic agent is administered in a sub-perceptible dose.

96. The method of any one of claims 90-95, wherein the notification informs a clinician's decision when to release the patient from a supervised facility.

97. The method of any one of claims 90-96, wherein the language sample is analyzed to derive the one or more language characteristics, wherein the one or more language characteristics comprises a measure of semantic proximity to one or more concepts related to an influence of a psychedelic agent.

98. The method of claim 97, wherein a measure of semantic proximity to one or more facets related to an influence of a psychedelic agent is positively correlated with the influence of the psychedelic therapy on the perception of the patient.

99. A method of screening a candidate for treatment with a psychedelic agent, the method comprising:
(i) obtaining a behavioral sample from the candidate, wherein the candidate has not begun treatment with a psychedelic agent;
(ii) deriving one or more behavioral characteristics from the behavioral sample; and
(iii) based on the one or more behavioral characteristics, determining a measure of risk, wherein the measure of risk correlates with a risk of precipitating or exacerbating psychosis, hypomania, or mania in the candidate.

100. The method of claim 99, further comprising sending a report to a third party.

101. The method of claim 100, wherein the third party is a clinical professional.

102. The method of claim 101, wherein the clinical professional is a physician, pharmacist, administrative professional, nurse, support professional, or caretaker.

103. The method of claim 100, wherein the third party is a computing platform.

104. The method of any one of claims 100-103, wherein the report informs a decision to prescribe or administer the psychedelic therapy.

105. The method of any one of claims 100-104, wherein the report informs a dosing regimen for the psychedelic therapy

106. A method of reducing a risk of developing psychosis, hypomania, or mania in a patient undergoing treatment with a psychedelic agent, the method comprising:
(i) obtaining a behavioral sample from the patient undergoing treatment with a psychedelic agent;
(ii) deriving one or more characteristics of the behavioral sample;
(iii) based on the one or more characteristics, determining a measure of risk, wherein the measure of risk correlates with the risk of precipitating or exacerbating psychosis, hypomania, or mania in the patient; and
(iv) based on the measure of risk, recommending whether to suspend the treatment with a psychedelic agent.

107. The method of claim 106, further comprising sending a report to a third party.

108. The method of claim 107, wherein the third party is a clinical professional.

109. The method of claim 108, wherein the clinical professional is a physician, pharmacist, administrative professional, nurse, support professional, or caretaker.

110. The method of claim 109, wherein the third party is a computing platform.

111. A method of assessing a risk of precipitating or exacerbating psychosis, hypomania, or mania in a patient undergoing treatment with a psychedelic agent, the method comprising:
(i) providing a plurality of measures of risk, each measure of risk derived from one or more characteristics of a behavioral sample obtained from the patient, wherein each measure of risk is associated with a different treatment time point; and
(ii) comparing two or more of the plurality of measure of risk to obtain a differential measure of risk, wherein the patient is identified as at risk of precipitating or exacerbating psychosis, hypomania, or mania if the differential measure of risk exceeds a predetermined threshold.

112. The method of claim 111, further comprising sending one or more reports to a third party.

113. The method of claim 112, wherein the third party is a clinical professional.

114. The method of claim 113, wherein the clinical professional is a physician, pharmacist, administrative professional, nurse, support professional, or caretaker.

115. The method of claim 114, wherein the third party is a computing platform.

116. The method of any one of claims 112-115, wherein the one or more reports recommends suspending the treatment with the psychedelic agent if the differential risk measure exceeds the predetermined threshold.

117. A method of providing a regimen of psychedelic therapy to a patient, the method comprising:
(i) providing a differential measure of risk obtained by comparing two or more measures of risk, each measure of risk derived from one or more behavioral characteristics of a behavioral sample obtained from the patient, wherein the one or more measures of risk correlates with the risk of precipitating or exacerbating psychosis, hypomania, or mania in the patient, and wherein each measure of risk is associated with a different treatment time point; and
(ii) suspending the psychedelic therapy if the differential measure of risk exceeds a predetermined threshold.

118. A method of administering a psychedelic agent to a patient in need thereof, the method comprising:
(i) obtaining one or more measures of risk derived from one or more behavioral characteristics of a behavioral sample obtained from the patient, wherein the one or more measures of risk correlates with the risk of precipitating or exacerbating psychosis, hypomania, or mania in the patient; and
(ii) administering the psychedelic agent if the measure of risk is below a predetermined threshold.

119. The method of any one of claims 99-118, further comprising, in response to determining that a candidate has a high measure of risk, prompting an EMA of the candidate.

120. The method of any one of claims 99-119, wherein the one or more behavioral characteristics are derived from a telephone record.

121. The method of claim 120, wherein the one or more behavioral characteristics derived from a telephone record comprise a number or frequency of outgoing calls or messages, a number or frequency of incoming calls or messages, a ratio between a number or frequency of outgoing calls or messages and a number or frequency of incoming calls or messages, a duration of one or more calls, a length of one or more messages, a number or frequency of newly added contacts, a number of changes in cell tower IDs, or a number of unique cell tower IDs.

122. The method of claim 121, wherein the number or frequency of outgoing calls or messages is positively correlated with the risk of developing hypomania or mania.

123. The method of claim 121, wherein the ratio between a number or frequency of outgoing calls or messages and a number or frequency of incoming calls or messages is positively correlated with the risk of developing hypomania or mania.

124. The method of claim 121, wherein the duration of one or more calls is positively correlated with the risk of developing hypomania or mania.

125. The method of claim 121, wherein the length of one or more messages is positively correlated with the risk of developing hypomania or mania.

126. The method of claim 121, wherein a number of unique phone numbers is positively correlated with the risk of developing hypomania or mania.

127. The method of any one of claims 99-126, wherein the one or more behavioral characteristics comprise a number or frequency of instances in which a mobile device screen is turned on.

128. The method of any one of claims 99-127, wherein the one or more behavioral characteristics comprise a measure of activity detected by a sensor.

129. The method of claim 128, wherein the sensor is an antenna on a mobile device.

130. The method of claim 128 or 129, wherein the sensor is in communication with a global positioning system (GPS).

131. The method of any one of claims 128-130, wherein the measure of activity is a measure of mobility.

132. The method of claim 131, wherein a high measure of mobility is positively correlated with the risk of developing hypomania or mania.

133. The method of claim 128, wherein the sensor is an accelerometer.

134. The method of claim 133, wherein the measure of activity comprises a measure of movement.

135. The method of claim 134, wherein a measure of movement is positively correlated with the risk of developing hypomania or mania.

136. The method of any one of claims 129-135, wherein the sensor is or is in communication with a wireless network hub.

137. The method of any one of claims 99-136, wherein the measure of risk is further based on one or more language characteristics derived from a language sample.

138. The method of claim 137, wherein the language sample is elicited by a digital prompt, a questionnaire, or a clinician administered interview.

139. The method of claim 137 or 138, wherein the language sample is a dream report, a description of a picture, a thematic apperception test, or a neutral text reading.

140. The method of any one of claims 137-139, wherein the language sample is obtained by passive acquisition.

141. The method of any one of claims 137-140, wherein the language sample is a text sample.

142. The method of any one of claims 137-141, wherein the language sample is an audio sample.

143. The method of claim 142, wherein the audio sample is analyzed to derive the one or more language characteristics, wherein the one or more language characteristics comprises one or more acoustic features.

144. The method of claim 143, wherein the one or more acoustic features are selected from the group consisting of a measure of irregular pitch, zero-crossing rate, kurtosis energy, HNR, MFCC, and frame energy.

145. The method of any one of claims 142-144, wherein the audio sample is transcribed into text.

146. The method of any one of claims 137-145, wherein the language sample is analyzed to derive the one or more language characteristics, wherein the one or more language characteristics comprises a measure of semantic coherence.

147. The method of claim 146, wherein a low measure of semantic coherence is positively correlated with the risk of developing psychosis.

148. The method of any one of claims 137-147, wherein the language sample is analyzed to derive the one or more language characteristics, wherein the one or more language characteristics comprises a measure of syntactic complexity.

149. The method of claim 148, wherein a low measure of syntactic complexity is positively correlated with the risk of developing psychosis.

150. The method of any one of claims 137-149, wherein the language sample is analyzed to derive the one or more language characteristics, wherein the one or more language characteristics comprises a measure of maximum phrase length.

151. The method of claim 150, wherein a high measure of maximum phrase length is positively correlated with the risk of developing hypomania or mania.

152. The method of any one of claims 137-151, wherein the language sample is analyzed to derive the one or more language characteristics, wherein the one or more language characteristics comprises a measure of lexicon breadth or depth.

153. The method of claim 152, wherein a high measure of lexicon breadth or depth is positively correlated with the risk of developing hypomania or mania.

154. The method of any one of claims 137-153, wherein the language sample is analyzed to derive the one or more language characteristics, wherein the one or more language characteristics comprises a measure of logorrhea.

155. The method of claim 154, wherein a high measure of logorrhea is positively correlated with the risk of developing hypomania or mania.

156. The method of any one of claims 137-155, wherein the language sample is analyzed to derive the one or more language characteristics, wherein the one or more language characteristics comprises a measure of psychometrics.

157. The method of claim 156, wherein the measure of psychometrics is latent inhibition.

158. The method of any one of claims 137-157, wherein the language sample is analyzed to derive the one or more language characteristics, wherein the one or more language characteristics comprises a measure of flight of thought.

159. The method of claim 158, wherein a high measure of flight of thought is positively correlated with the risk of developing hypomania or mania.

160. The method of any one of claims 137-159, wherein the language sample is analyzed to derive the one or more language characteristics, wherein the one or more language characteristics comprises a measure of recursiveness.

161. The method of claim 160, wherein a high measure of recursiveness is positively correlated with the risk of developing hypomania or mania.

162. The method of any one of claims 119-161, wherein the measure of risk is further based on a result of the EMA.

163. The method of claim 162, wherein the measure of risk is a measure of risk of precipitating or exacerbating hypomania or mania, and wherein the EMA is a psychopathology questionnaire configured to assess hypomania or mania.

164. The method of claim 163, wherein the EMA is the Hypomania/Mania Symptom Checklist (HCL-32), the Clinician-Administered Rating Scale for Mania (CARS-M), the Young Mania Rating Scale (YMRS), or an equivalent variant thereof.

165. The method of claim 162, wherein the measure of risk is a measure of risk of precipitating or exacerbating psychosis, and wherein the EMA is a psychopathology questionnaire configured to assess psychosis.

166. The method of claim 165, wherein the EMA is the psychosis screening questionnaire, the STEPI, the CBQp, or an equivalent variant thereof.

167. The method of any one of claims 1-166, wherein the measure of risk is determined using a machine learning algorithm.

168. The method of any one of claims 1-167, wherein the measure of risk is determined using a cluster model.

169. The method of any one of claims 1-168, wherein the measure of risk is determined based on a change of one or more of the characteristics relative to a reference characteristic.

170. The method of claim 169, wherein the reference characteristic is obtained from the patient at an earlier time point.

171. The method of claim 169, wherein the reference characteristic is a predetermined threshold.

172. The method of any one of claims 1-171, wherein the psychedelic therapy is being administered for treatment of condition.

173. The method of claim 172, wherein the condition is a chronic condition.

174. The method of claim 172 or 173, wherein the condition is an inflammatory-related condition.

175. The method of any one of claims 172-174, wherein the condition is Alzheimer's disease.

176. The method of claim 172-175, wherein the condition is depression.

177. The method of claim 176, wherein the depression is major depression, melancholic depression, or atypical depression.

178. The method of claim 176, wherein the depression is dysthymia.

179. The method of claim 172 or 173, wherein the condition is a psychological disorder selected from the group consisting of an anxiety disorder, an addiction, a compulsive behavior disorder, or a symptom thereof.

180. The method of any one of claims 1-171, wherein the psychedelic therapy is being administered for improvement of mood or enhancement of performance.

181. The method of any one of claims 1-171, wherein the psychedelic therapy is being administered for treatment of stress, treatment of anxiety, treatment of addiction, treatment of depression, or treating of a compulsive behavior.

182. The method of any one of claims 1-171, wherein the psychedelic therapy is being administered for treatment to improve the mental well-being of a patient.

183. The method of any one of claims 1-171, wherein the psychedelic therapy is being administered to reduce the risk of occurrence or reoccurrence of a psychophathology.

184. The method of any one of claims 1-183, wherein the psychedelic therapy is part of a complex therapy, wherein the patient is additionally being treated with a psychotherapy.

185. The method of claim 184, wherein the psychotherapy comprises behavioral activation therapy, talk therapy, existential therapy, and/or self-actualization therapy.

186. The method of claim 185, wherein the behavioral activation therapy is brief behavioral activation for depression (BATD).

187. The method of any one of claims 184-186, wherein the complex therapy is provided to the patient in a specialized treatment facility.

188. The method of claim 172, wherein the condition is a neurodegenerative condition.

189. The method of claim 188, wherein the patient has undergone a cognitive assessment.

190. The method of claim 188, further comprising conducting a cognitive assessment on the patient.

191. The method of claim 189 or 190, further comprising discontinuing treatment based on a result of the cognitive assessment, wherein the negative result is associated with drug-related brain decline.

192. The method of claim 189 or 190, further comprising discontinuing treatment based on behavioral characteristic derived from an interaction between the patient and a device.

193. The method of any one of claim 192, wherein the cognitive assessment is selected from the group consisting of a mini-mental state examination (MMSE), Montreal cognitive assessment (MOCA), and Alzheimer's Disease assessment scale-cognitive subscale (ADAS-Cog).

194. The method of any one of claims 1-193, wherein the psychedelic therapy comprises administration of an agent selected from the group consisting of a 5-HT_{2A} receptor agonist, an empathogenic agent, and a dissociative agent.

195. The method of claim 194, wherein the psychedelic therapy comprises administration of a 5-HT_{2A} receptor agonist.

196. The method of claim 195, wherein the 5-HT_{2A} receptor agonist is selected from lysergic acid diethylamide (LSD), psilocybin, DOI (±)-1-(2,5-dimethoxyphenyl)-2-aminopropane hydrochloride; (R)-DOI ((R)-1-(2,5-dimethoxy-4-iodophenyl)-2-aminopropane); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl) methylamine) ayahuasca; 3,4,5-trimethoxyphenethylamine (mescaline); 5-methoxy-N,N-dimethyltryptamine (5-meo-DMT); ibogaine; a compound of 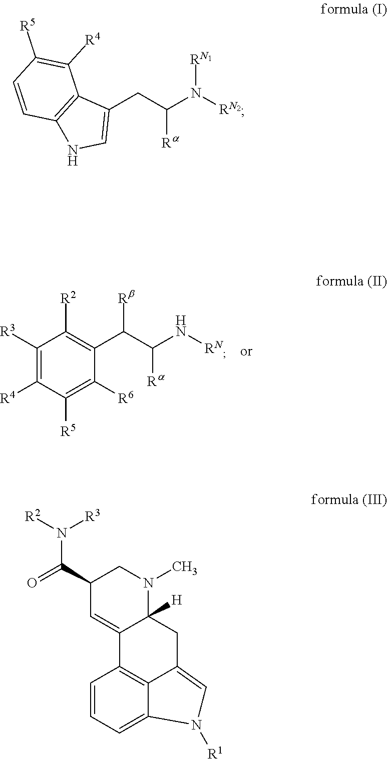
or a pharmaceutically acceptable salt thereof.

197. The method of claim 196, wherein the psychedelic agent is an empathogenic agent.

198. The method of claim 197, wherein the empathogenic agent is 3,4-methylenedioxymethamphetmine (MDMA).

199. The method of claim 194, wherein the psychedelic agent is a dissociative agent.

200. The method of claim 199, wherein the dissociative agent is ketamine or esketamine.

201. A software program configured for assessing a risk of precipitating or exacerbating psychosis, hypomania, or mania in a patient undergoing treatment with a psychedelic agent or a candidate for treatment with a psychedelic agent, the software program comprising computer-readable instructions for performing the method of any one of claims 1-200.

202. A software program configured for assessing a risk of precipitating or exacerbating psychosis, hypomania, or mania in a patient undergoing treatment with a psychedelic agent or a candidate for treatment with a psychedelic agent, the software program comprising computer-readable instructions for:
(i) obtaining one or more language and/or behavioral samples from the user;
(ii) deriving one or more language characteristics from the one or more language samples and/or one or more behavioral characteristics from the one or more behavioral samples; and based on the one or more language and/or behavioral characteristics, determining a measure of risk, wherein the measure of risk correlates with a risk of precipitating or exacerbating psychosis, hypomania, or mania in the candidate; and
(iii) reporting the measure of risk to the user and/or a third party.

203. The software program of claim 202, further comprising computer-readable instructions for receiving information regarding the treatment with the psychedelic agent, wherein the information is selected from the group consisting of psychedelic agent composition, a quantity of psychedelic agent prescribed, a dosing schedule, a quantity of psychedelic agent administered per dose, a frequency of doses administered, and a cumulative quantity of psychedelic agent administered.

204. The software program of claim 203, wherein the computer-readable instructions for receiving information regarding the treatment with the psychedelic agent are configured to receive the information from the patient, a clinician, or the third party.

205. The software program of any one of claims 202-204, wherein the computer-readable instructions for receiving information regarding the treatment with the psychedelic agent are further configured to store and/or report the information regarding the treatment with the psychedelic agent.

206. The software program of claim 205, wherein the computer-readable instructions for receiving information regarding the treatment with the psychedelic agent are configured to report all or a portion of the information to the patient.

207. The software program of claim 205, wherein the computer-readable instructions for receiving information regarding the treatment with the psychedelic agent are configured to report all or a portion of the information to the third party.

208. The software program of claim 207, wherein the computer-readable instructions for receiving information regarding the treatment with the psychedelic agent are configured to report all or a portion of the information to the third party upon detecting non-compliance by the patient.

209. A computer system for assessing a risk of precipitating or exacerbating psychosis, hypomania, or mania in a patient undergoing treatment with a psychedelic agent or a candidate for treatment with a psychedelic agent, the computer system comprising:
(i) a mobile device comprising one or more input mechanisms, a processor, and one or more output mechanisms; and
(ii) a software program readable by the processor, the software program comprising instructions for:
(a) using the one or more input mechanisms, obtaining one or more language and/or behavioral samples from the user;
(b) using the processor, deriving one or more language characteristics from the one or more language samples and/or one or more behavioral characteristics from the one or more behavioral samples; and based on the one or more language and/or behavioral characteristics, determining a measure of risk, wherein the measure of risk correlates with a risk of precipitating or exacerbating psychosis, hypomania, or mania in the candidate; and
(c) using the one or more output mechanisms, reporting the measure of risk to the user and/or a third party.

210. The computer system of claim 209, wherein the software program further comprises computer-readable instructions for receiving information regarding the treatment with the psychedelic agent, wherein the information is selected from the group consisting of psychedelic agent composition, a quantity of psychedelic agent prescribed, a dosing schedule, a quantity of psychedelic agent administered per dose, a frequency of doses administered, and a cumulative quantity of psychedelic agent administered.

211. The computer system of claim 210, wherein the computer-readable instructions for receiving information regarding the treatment with the psychedelic agent are configured to receive the information from the patient, a clinician, or the third party.

212. The computer system of any one of claims 209-211, wherein the computer-readable instructions for receiving information regarding the treatment with the psychedelic agent are further configured to store and/or report the information regarding the treatment with the psychedelic agent.

213. The computer system of claim 212, wherein the computer-readable instructions for receiving information regarding the treatment with the psychedelic agent are configured to report all or a portion of the information to the patient.

214. The computer system of claim 213, wherein the computer-readable instructions for receiving information regarding the treatment with the psychedelic agent are configured to report all or a portion of the information to the third party.

215. The computer system of claim 214, wherein the computer-readable instructions for receiving information regarding the treatment with the psychedelic agent are configured to report all or a portion of the information to the third party upon detecting non-compliance by the patient.

216. The software program of any one of claims 201-208 or the computer system of any one of claims claim 209-215, further comprising a psychotherapy application, wherein the psychotherapy application is configured to provide psychotherapy to the patient or candidate.

217. The software program or computer system of claim 216, wherein the psychotherapy is provided via telemedicine.

218. The software program or computer system of claim 216 or 217, wherein the psychotherapy is behavioral activation therapy.
